(19) United States
(12) Patent Application Publication
(10) Pub. No.: US 2020/0352912 A1
(43) Pub. Date: Nov. 12, 2020

(54) ANTIMICROBIAL COMPOSITIONS AND METHODS OF USING THE SAME

(71) Applicant: The Board Of Supervisors Of Louisiana State University And Agricultural And Mechanical College, Baton Rouge, LA (US)

(72) Inventor: Mairi C. Noverr, New Orleans, LA (US)

(21) Appl. No.: 16/965,227

(22) PCT Filed: Jan. 25, 2019
(86) PCT No.: PCT/US2019/015223

§ 371 Date: Jul. 27, 2020

Related U.S. Application Data
(60) Provisional application No. 62/622,589, filed on Jan. 26, 2018.

(51) Int. Cl. A61K 31/4196; A61P 31/04; A61L 2/16

(52) U.S. Cl. CPC A61K 31/4196; A61K 9/0014; A61L 2/16; A61P 31/04

(57) ABSTRACT

This invention is directed to antimicrobial compositions and methods of using the same to treat or prevent infection. A method of treating microbial infection in a subject, the method comprising administering to the subject a therapeutically effective amount of an EP4 receptor antagonist, wherein the EP4 receptor antagonist inhibits the growth of the microorganism.

This application claims priority from U.S. Provisional Application No. 62/622,589, filed on Jan. 26, 2018, the entire contents of which is incorporated herein by reference.

### GOVERNMENT INTERESTS

This invention was made with government support under Grant No. R01 AI116025 awarded by the National Institutes of Health. The government has certain rights in the invention.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### FIELD OF THE INVENTION

This invention is directed to antimicrobial compositions and methods of using the same to treat or prevent infection.

### BACKGROUND OF THE INVENTION

Intra-abdominal infection (IAI) is a broad term used to describe infections that occur as a result of the perforation of the gastrointestinal tract. There is currently a 77% mortality rate associated with polymicrobial IAI involving fungal and bacterial species, a rate that far exceeds that of bacterial monomicrobial infections (20%). A plethora of microorganisms can cause IAI, however, two microorganisms that are frequently co-isolated are the fungal pathogen, Candida albicans and the pathogenic bacterium, Staphylococcus aureus. In patients with intra-abdominal perforations, isolation of C. albicans alone is indicative of high mortality risk. Using animal models, it was demonstrated that co-infection with S. aureus raises the mortality rate even further as a lethal synergistic association exists between these two pathogens.

### SUMMARY OF THE INVENTION

Embodiments of the invention provide a method of treating microbial infection in a subject, the method comprising administering to the subject a therapeutically effective amount of an EP4 receptor antagonist, wherein the EP4 receptor antagonist inhibits the growth of the microorganism.

Embodiments of the invention still further provide a method of preventing microbial infection in a subject, the method comprising administering to the subject a prophylactically effective amount of an EP4 receptor antagonist, wherein the EP4 receptor antagonist inhibits the growth of the microorganism.

In embodiments, the EP4 receptor antagonist comprises L-161, 982 (CAS No. 147776-06-5) or an analog thereof.

In embodiments, the EP4 receptor antagonist comprises a compound of the chemical structure:

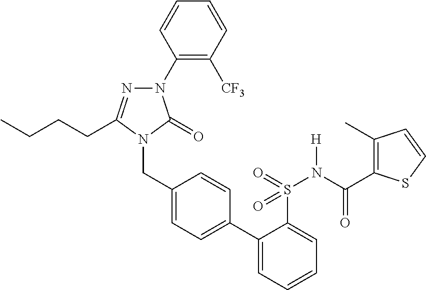 [SMILES: CCCCc1nn(-c2ccccc2C(F)(F)F)c(=O)n1Cc1ccc(-c2ccccc2S(=O)(=O)NC(=O)c2sccc2C)cc1]

In embodiments, the EP4 receptor antagonist prevents or inhibits the growth of a microorganism.

In embodiments, the EP4 receptor antagonist is administered prophylactically.

In embodiments, the infection comprises a mono-species infection or a poly-species infection. For example, the poly-species infection comprises a dual-species infection. For example, the poly-species infection comprises S. aureus and C. albicans.

In embodiments, the infection comprises a Staphylococcus infection, a Streptococcus infection, or both. For example, the Staphylococcus infection comprises Staphylococcus aureus or Staphylococcus epidermidis. For example, the Streptococcus infection comprises Streptococcus mutans. In embodiments, the infection comprises methicillin-resistant S. aureus (MRSA).

In embodiments, the infection further comprises Candida, for example C. albicans.

In embodiments, the microbial infection causes sepsis or peritonitis.

In embodiments, the microbial infections comprises a bloodstream infection (i.e., bacteremia).

In some embodiments, the therapeutically effective amount is at least about 0.1 mg/kg body weight, at least about 0.25 mg/kg body weight, at least about 0.5 mg/kg body weight, at least about 0.75 mg/kg body weight, at least about 1 mg/kg body weight, at least about 2 mg/kg body weight, at least about 3 mg/kg body weight, at least about 4 mg/kg body weight, at least about 5 mg/kg body weight, at least about 6 mg/kg body weight, at least about 7 mg/kg body weight, at least about 8 mg/kg body weight, at least about 9 mg/kg body weight, at least about 10 mg/kg body weight, at least about 15 mg/kg body weight, at least about 20 mg/kg body weight, at least about 25 mg/kg body weight, at least about 30 mg/kg body weight, at least about 40 mg/kg body weight, at least about 50 mg/kg body weight, at least about 75 mg/kg body weight, at least about 100 mg/kg body weight, at least about 200 mg/kg body weight, at least about 250 mg/kg body weight, at least about 300 mg/kg body weight, at least about 3500 mg/kg body weight, at least about 400 mg/kg body weight, at least about 450 mg/kg body weight, at least about 500 mg/kg body weight, at least about 550 mg/kg body weight, at least about 600 mg/kg body weight, at least about 650 mg/kg body weight, at least about 700 mg/kg body weight, at least about 750 mg/kg body weight, at least about 800 mg/kg body weight, at least about 900 mg/kg body weight, or at least about 1000 mg/kg body weight. For example, an effective amount can comprise a dose of about 10 mg/kg body weight.

In embodiments, the therapeutically effective amount is at least about 0.1 μg/ml, at least about 0.5 μg/ml, at least about 1 μg/ml, at least about 2 μg/ml, at least about 3 μg/ml, at least about 4 μg/ml, at least about 5 μg/ml, at least about 6 μg/ml, at least about 6.3 μg/ml, at least about 7 μg/ml, at least about 8 μg/ml, at least about 9 μg/ml, at least about 10 μg/ml, at least about 20 μg/ml, at least about 30 μg/ml, at least about 40 μg/ml, or at least about 50 μg/ml.

For example, the EP4 receptor antagonist is administered at a dose of about 10 mg/kg body weight.

For example, the EP4 receptor antagonist is administered at a dose of about 6.3 μg/ml.

In embodiments, the EP4 receptor antagonist is provided in a pharmaceutically acceptable composition or as a pharmaceutically acceptable salt.

In embodiments, the EP4 receptor antagonist is administered locally or systemically. In embodiments, the EP4 receptor antagonist can be administered nasally, orally, by infusion or bolus injection, by absorption through epithelial or mucocutaneous linings (e.g., oral, rectal, vaginal, and intestinal mucosa, etc.), intradermal, intramuscular, intraperitoneal, intravenous, subcutaneous, infusion, intranasal, epidural, oral, sublingual, intracerebral, intravaginal, transdermal, rectal, by inhalation, or topical, particularly to the ears, nose, eyes, or skin

Further, embodiments of the invention provide a method of inhibiting the growth of a microorganism, the method comprising contacting the microorganism with an EP4 receptor antagonist for a period of time sufficient to inhibit the growth of the microorganism. For example, the period of time can be 1 second, 10 seconds, 30 seconds, 1 minute, 5 minutes, 15 minutes, 30 minutes, 1 hour, or longer than 1 hour.

In embodiments, the EP4 receptor antagonist comprises L-161, 982 (CAS No. 147776-06-5) or an analog thereof.

In embodiments, the EP4 receptor antagonist comprises a compound of the chemical structure:

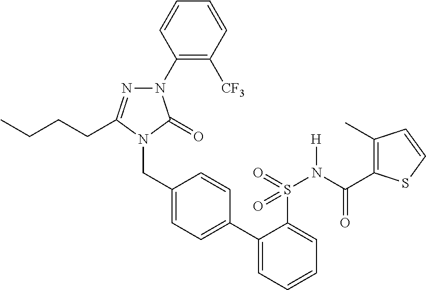 [SMILES: CCCCc1nn(-c2ccccc2C(F)(F)F)c(=O)n1Cc1ccc(-c2ccccc2S(=O)(=O)NC(=O)c2sccc2C)cc1]

Still further, embodiments of the invention provide a method of preventing the contamination of a medical device with a microorganism, the method comprising incorporating into and/or coating the medical device with an antimicrobial composition comprising an EP4 receptor antagonist.

Embodiments of the invention also provide a method of decreasing formation of a microbial biofilm, the method comprising contacting the microorganism with an EP4 receptor antagonist for a period of time sufficient to decrease the biofilm formation.

In embodiments, the method prevents the formation of a biofilm on the medical device.

In embodiments, the microorganism comprises a bacterium.

In embodiments, the bacterium comprises Staphylococcus aureus, Staphylococcus epidermidis, or Streptococcus mutans.

In embodiments, the EP4 receptor antagonist is administered topically or systemically.

Aspects of the invention are further directed towards a method of preventing biofilm formation comprising applying to a surface of interest an antimicrobial composition comprising an EP4 receptor antagonist. For example, the surface comprises the surface of a medical device. For example, the medical device is coated with the EP4 receptor antagonist.

In embodiments, the composition comprises a disinfectant, such as an aerosol disinfectant.

Still further, aspects of the invention are directed towards a method of preventing biofilm formation comprising incorporating into a material, such as a bio-compatible material, an EP4 receptor antagonist. Non-limiting examples of biocompatible materials comprise PGA, PLA, PCLA, PLGA, poly(L-lactic acid), polybutylate, silicone, biodegradable calcium phosphate, porous 4-fluorinated ethylene resin, polyproplylene, amylose, cellulose, synthetic DNA, polyesters, and the like

Aspects of the invention are also directed towards a method of disrupting a biofilm comprising contacting the microbial biofilm with an EP4 receptor antagonist for a period of time sufficient to disrupt the pre-formed microbial biofilm.

In embodiments, the biofilm comprises a mono-species biofilm or a poly-species biofilm. For example, the poly-species biofilm comprises a dual-species bio-film. For example, the biofilm comprises Staphylococcus, Streptococcus, or both.

In embodiments, the biofilm further comprises Candida, such as C. albicans. In embodiments, the biofilm comprises S. aureus and C. albicans.

In embodiments, the microorganism comprises a bacterium, for example a gram-positive bacterium.

In embodiments, the bacterium comprises antibiotic-resistant bacterium, for example a bacterium resistant to methicillin.

Other objects and advantages of this invention will become readily apparent from the ensuing description.

### BRIEF DESCRIPTION OF THE FIGURES

FIG. 1 shows L-161, 982 inhibits the growth of S. aureus strains. Effect of COX enzyme inhibitors or PGE2 EP receptor antagonists on microbial growth. The antimicrobial activity of selective COX enzyme inhibitor or PGE2 EP receptor antagonist on the growth of C. albicans DAY185 (A), S. aureus NRS383 (B) and MRSA and MSSA clinical isolates (C). Growth of C. albicans and S. aureus was monitored for up to 24 h in medium alone or supplemented with DMSO or EP receptor antagonists or COX enzyme inhibitor at physiologically relevant concentrations. Data shown are representative of three independent experiments.

FIG. 2 shows growth inhibition kinetics of L-161, 982. Growth of S. aureus NRS383 was monitored for up to 24 h in medium alone or supplemented with DMSO or 50 μg/ml L-161, 982. After 6 h of coincubation, fresh L-161, 982 was added to growth medium (*). Data shown are representative of three independent experiments.

FIG. 3 shows antimicrobial activity of L-161, 982 has broad activity specific against Gram-positive bacteria. Bacterial growth was monitored for 6 h in medium alone or supplemented with 50 μg/ml of L-161, 982. Data shown is representative of three independent experiments.

FIG. 4 shows L-161, 982 prevents MRSA biofilm formation. The effect of L-161,982 on biofilm formation was tested. Mono- and dual-species C. albicans and/or S. aureus biofilms were grown on 96-well plates for 24 h with the indicated concentration of L-161, 982. After incubation, metabolic activity of biofilms was quantified by the XTT assay (A) and then biofilms were disrupted to determine live colony forming units (CFU) (B). The results are cumulative of three independent experiments with duplicate samples. Statistics was performed using One-way ANOVA with the Tukey post-hoc test for the XTT assay. The Mann-Whitney U test was used to analyze CFU data. Significance is indicated as follows: ns—not significant, *P<0.05; *** P<0.001. Error bars represent standard deviation.

FIG. 5 shows the growth inhibitory effect of L-161, 982 on MRSA is not due to oxidative stress. Growth of S. aureus was monitored for up to 6 h in medium alone or medium supplemented with indicated antioxidant(s) and/or L-161, 982. Data shown are representative of two independent experiments.

FIG. 6 shows effect of L-161, 982 on survival and microbial burden. Mice were infected intraperitoneally with 0.2 ml of the indicated inoculum of S. aureus NRS383 in 3% hog gastric mucin/PBS. One group of mice/inoculum received either the vehicle or L-161, 982 at a dose of 10 mg/kg by i.p. injection as indicated in Materials and Methods. (A) Mortality was assessed using Kaplan Meier test (*P<0.05; **P<0.01; ***P<0.001; ns—not significant; n=15/group) compared to control groups. (B) Microbial burden in peritoneal lavage fluid and spleen was assessed between untreated (control) and treated (L-161, 982) groups 24 and 48 h post-inoculation (n=6 to 8/group). Values are plotted as the median CFU and compared using the Mann-Whitney U test (**P<0.01; ***P<0.001). Data shown are representative of three replicate experiments.

FIG. 7 shows antibacterial effect of L-161, 982 on MRSA enhances survival in a mouse model of C. albicans-S. aureus polymicrobial intra-abdominal infection. Effect of inhibition of EP4 receptor signaling on survival. Mice received either vehicle or EP4 receptor antagonist at a dose of 10 mg/kg by i.p. injection as indicated in Materials and Methods. Mortality was assessed using Kaplan Meier test (***P<0.001; n=20/group) compared to control groups. Data shown are representative of three replicate experiments.

FIG. 8 shows chemical structure of L-161, 982 (N-[[4′-[[3-Butyl-1,5-dihydro-5-oxo-1-[2-(trifluoromethyl)phenyl]-4H-1,2,4-triazol-4-yl]methyl][1,1′-biphenyl]-2-yl] sulfonyl]-3-methyl-2-thiophenecarboxamide). Highlighted is the sulfonamide functional group.

FIG. 9 shows effect of L-161, 982 on pre-formed mono/dual-species biofilm. The effect of L-161, 982 on pre-formed mature biofilms was tested. Mono- and dual-species C. albicans and/or S. aureus biofilms were grown on 96-well plates for 24 h before being treated with the indicated concentration of L-161, 982 for a further 24 h. After treatment, metabolic activity of biofilms was quantified by the XTT assay (A) and then biofilms were disrupted to determine live colony forming units (CFU) (B). The results are cumulative of three independent experiments with duplicate samples. Statistics were performed using One-way ANOVA with the Tukey post-hoc test for the XTT assay. The Mann-Whitney U test was used to analyze CFU data. Significance is indicated as follows: ns—not significant, *P<0.05; *** P<0.001. Error bars represent standard deviation.

FIG. 10 shows selective and non-selective COX enzyme inhibitors and EP receptor antagonists.

FIG. 11 shows chemical structure of L-181, 982 N-4′-3-Butyl-1,5-dihydro-5-oxo-1-2-trifluoromethylphenyl-4H-1,2,4-triazol-4-ylmethyl1,1′-biphenyl-2-ylsulfonyl-3-methyl-2-thiophenecarboxamide. Dotted red rectangle outlines the sulfonamide group and dotted blue outline denotes the urea group. Insets shows basic chemical structure of a sulfonamide group and urea group.

FIG. 12 shows growth inhibitory effects of L-161, 982 are not rescued by exogenous thymidine. S. aureus strain NRS383 was grown in cation-adjusted Mueller-Hinton II broth alone (control), with 50 μg/ml of L-161, 982 or with 40 μg/ml of SXT (positive control)±200 μg/ml of thymidine. Cultures were incubated at 37° C. non-shaking and aliquots were removed at various time points and cultured for growth (CFU). Results are expressed as % of control at each time point. Mean values from two independent experiments are shown.

FIG. 13 shows L-161, 982 does not cause cytoplasmic leakage. S. aureus was grown shaking at 37° C. in PBS alone, with 50 μg/ml L-161, 982 or with 4 μg/ml Chlorhexidine (positive control). The appearance of nucleic acid in culture supernatants was measured by OD260 at the indicated times. Data are expressed as change in absorbance at 260 nm compared to control (AA260). Data presented is cumulative of two independent experiments.

FIG. 14 shows growth inhibitory effects of L-161, 982 are not rescued by inhibitors of oxidative stress. (A) Effects of reactive oxygen species inhibitors. Growth of S. aureus was monitored for up to 6 h in medium alone or medium supplemented with 15 mM of Na-L-ascorbate, and/or 15 mM N-acetyl-L-cysteine, and/or 50 μg/ml L-161, 982. Data shown are representative of two independent experiments. (B) Effects of hydroxyl radical scavengers. Growth was monitored for 4 h in medium alone or medium supplemented with 50 μg/ml L-161, 982 or 1 mM H2O2 alone, plus 150 mM thiourea, or plus 500 μM 2,2′-dipyridyl.

FIG. 15 shows growth in the presence of L-161, 982 inhibits pigment production of S. aureus. Pigment (staphyloxanthin) levels were measured from cultures grown in Mueller-Hinton broth+/−L-161, 982 at 37° C. for 24 h. A) Macroscopic analysis of S. aureus pigment production. B) Pigment was extracted in methanol and optical density measured at 450 nm. Data shown are representative of two independent experiments. *** p<0.001 control vs. drug-treated.

FIG. 16 shows L-161, 982 treatment inhibits hemolytic activity of S. aureus. (A) Macroscopic analysis of S. aureus hemolytic activity. S. aureus was spotted on Mueller-Hinton agar containing sheep blood and/or various concentrations of L-161, 982, and incubated at 37 C for 24 h (0, 25 50, 100) or 72 h (200). (B) Width of the zone of RBC clearance was measured at 4 points around each S. aureus area of growth in 2 independent replicates. * p<0.05 control vs. drug-treated.

FIG. 17 shows L-161, 982 inhibits S. aureus ATP production. S. aureus was incubated with various concentrations of L-161,982 in a 96-well plate in triplicate for 5 h at 37° C. ATP production was measured in samples normalized to viable cell number using the BacTiter-Glo™ Assay (Promega) and measuring luminescence. Background luminescence from media alone samples was subtracted from experimental samples. Results represent the means+/−standard deviation. Data shown are representative of two independent experiments. ** p<0.01; *** p<0.001 control vs drug-treated.

### DETAILED DESCRIPTION OF THE INVENTION

Staphylococcus aureus is a clinically significant gram positive bacterial pathogen, causing a wide range of nosocomial infections ranging from pneumonia and skin or surgical site infections, to systemic bloodstream infections and sepsis. S. aureus is the leading cause of infection in critically ill and injury patients. There has been an alarming increase in methicillin-resistant S. aureus strains (MRSA) and multi-drug resistant (MDR) strains. These resistant strains are prevalent in both hospital-associated and community-acquired infections, which are associated with prolonged hospitalization and treatment costs and mortality rates of 20-50%. MRSA also causes polymicrobial infections, including systemic infections with the fungal pathogen Candida albicans.

Unexpectedly, we found that the EP4 receptor antagonist, L-161, 982, had direct growth-inhibitory effects on S. aureus in vitro at the physiological concentration required to block PGE2 interaction with EP4. This antimicrobial activity was observed with both methicillin-sensitive and methicillin-resistant S. aureus (MRSA). In addition, L-161, 982 inhibited S. aureus biofilm formation and had activity against pre-formed mature biofilms.

In vivo testing demonstrated that treatment of mice with L-161, 982 following intra-peritoneal inoculation with a lethal dose of MRSA significantly reduced microbial burden and enhanced survival. Furthermore, L-161, 982 protected mice against the synergistic lethality induced by co-infection with C. albicans and S. aureus. Importantly, administration of L-161,982 alone did not cause any overt signs of toxicity, with no morbidity or mortality observed in control animals. An alternative EP4 receptor antagonist exerted no antimicrobial or protective effects, indicating that the antimicrobial activity of L-161, 982 is independent of its effects on inhibiting EP4 receptor activity.

Thus, aspects of the invention are directed towards compositions and methods of preventing or treating a microbial infection in a subject, compositions and methods of preventing or inhibiting the growth of a microorganism, and compositions and methods of preventing biofilm formation on a surface of interest.

### Abbreviations and Definitions

Detailed descriptions of one or more embodiments are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in any appropriate manner.

The singular forms “a”, “an” and “the” include plural reference unless the context clearly dictates otherwise. The use of the word “a” or “an” when used in conjunction with the term “comprising” in the claims and/or the specification may mean “one,” but it is also consistent with the meaning of “one or more,” “at least one,” and “one or more than one.”

Wherever any of the phrases “for example,” “such as,” “including” and the like are used herein, the phrase “and without limitation” is understood to follow unless explicitly stated otherwise. Similarly “an example,” “exemplary” and the like are understood to be nonlimiting.

The term “substantially” allows for deviations from the descriptor that do not negatively impact the intended purpose. Descriptive terms are understood to be modified by the term “substantially” even if the word “substantially” is not explicitly recited.

The terms “comprising” and “including” and “having” and “involving” (and similarly “comprises”, “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b and c. Wherever the terms “a” or “an” are used, “one or more” is understood, unless such interpretation is nonsensical in context.

As used herein the term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

Aspects of the invention are directed towards compositions and methods of preventing or treating a disease or disorder in a subject by administering to the subject an effective amount of an EP4 receptor antagonist or derivatives or structural analogs thereof.

The terms “disorder” and “disease” are used herein interchangeably for a condition in a subject. A disorder is a disturbance or derangement that affects the normal function of the body of a subject. A disease is a pathological condition of an organ, a body part, or a system resulting from various causes, such as infection, genetic defect, or environmental stress that is characterized by an identifiable group of symptoms. For example, the disorder and/or disease comprises sepsis or peritonitis.

A disorder or disease can refer to an infection caused by a microorganism, which can also be referred to as a microbial infection. In an embodiment, the infection can comprise a mono-species infection, such as a Staphylococcus infection or a Streptococcus infection.

Staphylococcus is a genus of Gram-positive bacteria in the family Staphylococcaceae. Under the microscope, they appear spherical (cocci), and form in grape-like clusters. Staphylococcus can cause a wide variety of diseases in humans and animals through either toxin production or penetration. Staphylococcal toxins are a common cause of food poisoning, for they can be produced by bacteria growing in improperly stored food items. Staphylococcus includes at least 40 species. The taxonomy is based on 16s rRNA sequences, and most of the staphylococcal species fall into 11 clusters: S. aureus group (S. argenteus, S. aureus, S. schweitzeri, S. simiae), S. auricularis group (S. auricularis), S. carnosus group (S. carnosus, S. condimenti, S. massiliensis, S. piscifermentans, S. simulans), S. epidermidis group (S. capitis, S. caprae, S. epidermidis, S. saccharolyticus), S. haemolyticus group (S. devriesei, S. haemolyticus, S. hominis), S. hyicus-intermedius group (S. agnetis, S. chromogenes, S. cornubiensis, S. felis, S. delphini, S. hyicus, S. intermedius, S. lutrae, S. microti, S. muscae, S. pseudintermedius, S. rostri, S. schleiferi), S. lugdunensis group (S. lugdunensis), S. saprophyticus group (S. arlettae, S. caeli, S. cohnii, S. equorum, S. gallinarum, S. kloosii, S. leei, S. nepalensis, S. saprophyticus, S. succinus, S. xylosus), S. sciuri group (S. fleurettii, S. lentus, S. sciuri, S. stepanovicii, S. vitulinus), S. simulans group (S. simulans), and S. warneri group (S. pasteuri, S. warneri).

Streptococcus is a genus of gram-positive coccus (plural cocci), or spherical bacteria, that belongs to the family Streptococcaceae. Cell division in streptococci occurs along a single axis, so as they grow they tend to form pairs or chains that may appear bent or twisted. Contrast with that of staphylococci, which divide along multiple axes, thereby generating irregular grape-like clusters of cells. Many bacteria formerly grouped in the genus Streptococcus were separated out into the genera Enterococcus and Lactococcus. Currently, over 50 species are recognized in this genus. In addition to streptococcal pharyngitis (strep throat), certain Streptococcus species are responsible for many cases of pink eye, meningitis, bacterial pneumonia, endocarditis, erysipelas, and necrotizing fasciitis (the ‘flesh-eating’ bacterial infections). In the medical setting, the most important groups are the alpha-hemolytic streptococci S. pneumoniae and Streptococcus viridans group, and the beta-hemolytic streptococci of Lancefield groups A and B (also known as “group A strep” and “group B strep”).

TABLE 1
Medically relevant streptococci (not all are alpha hemolytic)

| Species | Host | Disease |
|---|---|---|
| S. pyogenes | human | pharyngitis, cellulitis |
| S. agalactiae | human, | neonatal meningitis and sepsis |
| | cattle | |
| S. dysgalactiae | human, | endocarditis, bacteremia, pneumonia, |
| | animals | meningitis, respiratory infections |
| S. bovis | human, | biliary or urinary tract infections, |
| | animals | endocarditis |
| S. anginosus | human, | subcutaneous/organ abscesses, |
| | animals | meningitis, respiratory infections |
| S. sanguinis | human | endocarditis, dental caries |
| S. suis | swine | meningitis |
| S. mitis | human | endocarditis |
| S. mutans | human | dental caries |
| S. pneumoniae | human | pneumonia |

In an embodiment, the infection can comprise a poly-species infection, such as a dual-species infection which comprises both Staphylococcus and Streptococcus microorganisms, such as an infection which comprises S. aureas and S. mutans. The skilled artisan will recognize that poly-species infections can further comprise one or more additional microorganisms. For example a poly-species infection comprising S. aureas can further comprises Candida species, such as C. albicans.

In embodiments, the infection can comprise an antibiotic-resistant infection, such as an infection cause by methicillin-resistant S. aureus (MRSA). Methicillin-resistant Staphylococcus aureus (MRSA) refers to a group of gram-positive bacteria that are genetically distinct from other strains of Staphylococcus aureus. MRSA is responsible for several difficult-to-treat infections in humans. MRSA is common in hospitals, prisons, and nursing homes, where people with open wounds, invasive devices such as catheters, and weakened immune systems are at greater risk of hospital-acquired infection. MRSA began as a hospital-acquired infection, but has become community-acquired as well as livestock-acquired. The terms HA-MRSA (healthcare-associated or hospital-acquired MRSA), CA-MRSA (community-associated MRSA) and LA-MRSA (livestock-associated) reflect this.

Embodiments can comprise contacting a cell or microorganism with an EP4 receptor antagonist for a period of time sufficient to inhibit the growth of the microorganism, thus preventing or treating a disease or disorder of the invention. For example, the growth of the microorganism can cause an infection in a subject. As another example, the growth of the microorganism can cause a biofilm to form or grow on the surface of an object.

Non-limiting examples of cells to be contacted with the EP4 receptor antagonist includes bacterial cells, yeast cells, protozoan cells, and cells infected with a viral or other pathogen. Representative pathogens include gram positive pathogens, such as gram positive bacteria. Representative bacteria include but are not limited to Staphylococcus sp., Streptococcus sp., Candida sp., Legionella sp., P. aeruginosa, H. influenzae, V. cholerae, Yersinia pestis, Escherichia coli, Listeria spp., and Clostridium spp. Alternatively, the cell to be contacted is an animal cell, such as a mammalian cell, or more specifically, a human cell. The cell may be from a particular tissue or cell line, such as an epithelial cell.

Prostaglandin E₂ receptor 4 (EP₄) is a prostaglandin receptor for prostaglandin E2 (PGF₂) encoded by the PTGER4 gene in humans. It is one of four identified EP receptors, the others being EP₁, EP₂, and EP₃, all of which bind with and mediate cellular responses to PGE₂. Aspects of the invention are directed towards EP4 receptor antagonists and uses thereof, such as to treat disease. The term “antagonist” can refer to any compound including a protein, polypeptide, peptide, antibody, antibody fragment, large molecule, or small molecule (less than 10 kD), that decreases the activity, activation or function of another molecule. For example, the EP4 receptor antagonist comprises L-161, 982 (CAS No. 147776-06-5). As another example, the EP4 receptor antagonist comprises analogs or derivatives of L-161,982.

L-161,982 is a potent and selective EP₄ receptor antagonist. It demonstrates selective binding to human EP₄ receptors with a Kᵢ value of 0.024 μM compared to other receptors of the prostanoid family, EP₁, EP₂, EP₃, DP, FP, and IP, with Kᵢ values of 17, 23, 1.9, 5.1, 5.6, and 6.7 μM, respectively (Machwate, M., Harada, S., Leu, C. T., et al. Prostaglandin receptor EP4 mediates the bone anabolic effects of PGE2 Molecular Pharmacology 60(1), 36-41 (2001)). L-161,982 at 10 mg/kg/day suppresses PGE₂-stimulated bone formation in young rats and at 100 nM reverses the anti-inflammatory action of PGE₂ in LPS-activated human macrophages (Takayama, K., Garcia-Gardeña, G., Sukhova, G. K., et al. Prostaglandin E2 suppresses chemokine production in human macrophages through the EP4 receptor The Journal of Biological Chemistry 277(46), 44147-44154 (2002)). At 10 μM L-161982 blocks PGE₂-induced cell proliferation in HCA-7 colon cancer cells (Cherukuri, D. P., Chen, X. B. O., Goulet, A. C., et al. The EP4 receptor antagonist, L-161,982, blocks prostaglandin E2-induced signal transduction and cell proliferation in HCA-7 colon cancer cells Experimental Cell Research 313, 2969-2979 (2007)).

L-161,982 comprises a compound of the chemical structure:

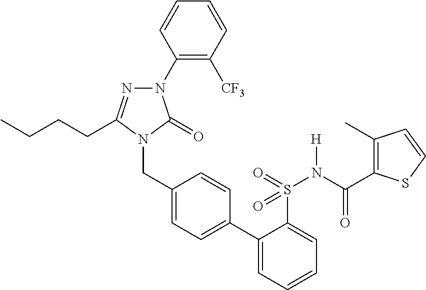 [SMILES: CCCCc1nn(-c2ccccc2C(F)(F)F)c(=O)n1Cc1ccc(-c2ccccc2S(=O)(=O)NC(=O)c2sccc2C)cc1]

Embodiments of the invention can comprise derivatives or analogs of an EP4 receptor antagonist, such as L-161,982. A derivative is a compound that can be imagined to arise or actually be synthesized from a parent compound by replacement of one atom with another atom or group of atoms. A “derivative” refers to a molecule that shares substantial structural similarity to its parent molecule. A derivative can be produced by chemical modifications using techniques known to those of skill in the art. For example, such derivatives may have improved solubility, improved activity, or longer half-lives, for example.

A structural analog, also known as a chemical analog or simply an analog, is a compound having a structure similar to that of another compound, but differing from it in respect to a certain component. A structural analog can be produced by chemical modifications using techniques known to those of skill in the art. For example, such analogs may have improved solubility, improved activity, or longer half-lives, for example.

An “effective amount” refers to, for example, the amount of a therapy, such as an EP4 receptor antagonist, which is sufficient to reduce or ameliorate the severity and/or duration of a disorder or one or more symptoms thereof, prevent the advancement of a disorder, cause regression of a disorder, prevent the recurrence, development, onset or progression of one or more symptoms associated with a disorder, detect a disorder, or enhance or improve the prophylactic or therapeutic effect(s) of another therapy (e.g., prophylactic or therapeutic agent). For example, an effective amount of an EP4 receptor antagonist can be administered prophylactically, such as to prevent a bacterial infection in a subject.

In some embodiments, the therapeutically effective amount is at least about 0.1 mg/kg body weight, at least about 0.25 mg/kg body weight, at least about 0.5 mg/kg body weight, at least about 0.75 mg/kg body weight, at least about 1 mg/kg body weight, at least about 2 mg/kg body weight, at least about 3 mg/kg body weight, at least about 4 mg/kg body weight, at least about 5 mg/kg body weight, at least about 6 mg/kg body weight, at least about 7 mg/kg body weight, at least about 8 mg/kg body weight, at least about 9 mg/kg body weight, at least about 10 mg/kg body weight, at least about 15 mg/kg body weight, at least about 20 mg/kg body weight, at least about 25 mg/kg body weight, at least about 30 mg/kg body weight, at least about 40 mg/kg body weight, at least about 50 mg/kg body weight, at least about 75 mg/kg body weight, at least about 100 mg/kg body weight, at least about 200 mg/kg body weight, at least about 250 mg/kg body weight, at least about 300 mg/kg body weight, at least about 3500 mg/kg body weight, at least about 400 mg/kg body weight, at least about 450 mg/kg body weight, at least about 500 mg/kg body weight, at least about 550 mg/kg body weight, at least about 600 mg/kg body weight, at least about 650 mg/kg body weight, at least about 700 mg/kg body weight, at least about 750 mg/kg body weight, at least about 800 mg/kg body weight, at least about 900 mg/kg body weight, or at least about 1000 mg/kg body weight. For example, an effective amount can comprise a dose of about 10 mg/kg body weight.

In embodiments, the therapeutically effective amount is at least about 0.1 μg/ml, at least about 0.5 μg/ml, at least about 1 μg/ml, at least about 2 μg/ml, at least about 3 μg/ml, at least about 4 μg/ml, at least about 5 μg/ml, at least about 6 μg/ml, at least about 6.3 μg/ml, at least about 7 μg/ml, at least about 8 μg/ml, at least about 9 μg/ml, at least about 10 μg/ml, at least about 20 μg/ml, at least about 30 μg/ml, at least about 40 μg/ml, or at least about 50 μg/ml.

The dosage can vary depending upon known factors such as the pharmacodynamic characteristics of the active ingredient and its mode and route of administration; time of administration of active ingredient; age, sex, health and weight of the recipient; nature and extent of symptoms; kind of concurrent treatment, frequency of treatment and the effect desired; and rate of excretion. These amounts can be readily determined by the skilled artisan.

The term “treating” can refer to partially or completely alleviating, ameliorating, improving, relieving, delaying onset of, inhibiting progression of, reducing severity of, and/or reducing incidence of one or more symptoms, features, or clinical manifestations of a particular disease, disorder, and/or condition. For example, “treating” an infection can refer to preventing, inhibiting, or reducing the growth of a microorganism. Treatment can be administered to a subject who does not exhibit signs of a disease, disorder, and/or condition (e.g., prior to an identifiable disease, disorder, and/or condition), and/or to a subject who exhibits only early signs of a disease, disorder, and/or condition for the purpose of decreasing the risk of developing pathology associated with the disease, disorder, and/or condition. In some embodiments, treatment comprises administering to a subject infected with a microorganism a therapeutically effective amount of an EP4 receptor antagonist, such as L-161,982.

The terms “prevent,” “preventing,” and “prevention” refer herein to the inhibition of the development or onset of a disorder or the prevention of the recurrence, onset, or development of one or more symptoms of a disorder in a subject resulting from the administration of a therapy (e.g., a prophylactic or therapeutic agent), or the administration of a combination of therapies (e.g., a combination of prophylactic or therapeutic agents).

As used herein, to “block” or “inhibit” a molecule, signal, or a receptor, such as the EP4 receptor, can refer to interfering with the binding of, or activation of the molecule, signal, or a receptor as detected by a test recognized in the art (such as binding assays). Blockage or inhibition may be partial or total, resulting in a reduction, increase, or modulation in the activation of the molecule, signal, or a receptor as detected by a test recognized in the art.

The terms “subject” and “patient” are used interchangeably throughout this disclosure. The terms refer to an animal, such as a human. For example, the terms can refer to a mammal including, but not limited to, a non-primate (e.g., a cow, pig, bird, sheep, goat, horse, cat, dog, rat, and mouse) and a primate (e.g., a monkey, such as a cynomolgous monkey, a chimpanzee, and a human). For example, the subject can be a non-human animal such as a bird (e.g., a quail, chicken, or turkey), a farm animal (e.g., a cow, horse, pig, or sheep), a pet (e.g., a cat, dog, or guinea pig), or laboratory animal (e.g., an animal model for a disorder). In particular, the subject according to the invention is a human (e.g., an infant, child, adult, or senior citizen).

In embodiments, the EP4 receptor antagonist reduces the growth of a microorganism. The reduction in microorganism growth can be indicative of the reduction in or inhibition of an infection in a subject. In some embodiments, the growth of microorganism can be determined by measuring the microorganism, such as bacteria, in a biological sample by using, for example, techniques known to the skilled artisan. In other embodiments, the presence or growth of a microorganisms is measured by detecting the presence of antigens of the microorganism in a biological sample. The biological sample can be blood, serum, sputum, lacrimal secretions, semen, urine, vaginal secretions, or a tissue sample. For example, an antibody to S. aureus components can be used as a test for colonization/infection in a subject afflicted with a bacterial infection, wherein the presence of Staphylococcus antigens is detected in a biological sample, such as blood. These antibodies can be generated according to methods well established in the art or can be obtained commercially (for example, from Abeam, Cambridge, Mass.; Cell Sciences Canton, Mass.; Novus Biologicals, Littleton, Colo.; or GeneTex, San Antonio, Tex.). The reduction in the growth of a microorganism can also be measured by chest x-rays, or by a pulmonary function test (PFT), such as spirometry or forced expiratory volume (FEV).

If the EP4 receptor antagonist is to be administered to a subject, it will be in the form of a pharmaceutically acceptable composition or formulation as described herein, wherein the composition or formulation is free of toxicity, which satisfies FDA requirements (see, for example, Remington: The Science and Practice of Pharmacy, 201 ed., Lippincott Williams & Wilkins, 2000; U.S. Pat. No. 6,030,604). Such an EP4 receptor antagonist composition, comprising compounds as described herein or pharmaceutically acceptable salts, can be administered to a subject afflicted with an infection or is at risk of developing an infection. Administration can occur alone or with other therapeutically effective composition(s) (e.g., additional antibiotics) either simultaneously or at different times.

The EP4 receptor antagonist can be administered to the subject one time (e.g., as a single injection or deposition). Alternatively, administration can be once or twice daily to a subject in need thereof for a period of time, such as from about 2 to about 28 days, or from about 7 to about 10 days, or from about 7 to about 15 days. It can also be administered once or twice daily to a subject for a period of time, such as 1, 2, 3, 4, 5, 6, 7, 8, 9, 10, 11, 12 times per year, or a combination thereof.

Formulations can include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient, which can be combined with a carrier material to produce a single dosage form, will generally be that amount of the compound that produces a therapeutic effect. Generally, out of one hundred percent, this amount will range from about 1 percent to about ninety-nine percent of active ingredient, e.g., from about 5 percent to about 70 percent, e.g., from about 10 percent to about 30 percent.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

The pharmaceutical composition can optionally comprise a suitable amount of a physiologically acceptable excipient. Non-limiting examples of physiologically acceptable excipients can be liquids, such as water and oils, including those of petroleum, animal, vegetable, or synthetic origin, such as peanut oil, soybean oil, mineral oil, sesame oil and the like; saline; gum acacia; gelatin; starch paste; talc; keratin; colloidal silica; urea and the like. In addition, auxiliary, stabilizing, thickening, lubricating, and coloring agents can be used. For example, the EP4 receptor antagonist composition and physiologically acceptable excipient are sterile when administered to a subject (such as an animal; for example a human). The physiologically acceptable excipient should be stable under the conditions of manufacture and storage and should be preserved against the contaminating action of microorganisms.

Water is a useful excipient when the compound or a pharmaceutically acceptable salt of the compound is administered intravenously. Saline solutions and aqueous dextrose and glycerol solutions can also be employed as liquid excipients, particularly for injectable solutions. Suitable physiologically acceptable excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

The pharmaceutical composition can be administered to the subject by any effective route, for example, systemically, topically, orally, by infusion or bolus injection, by absorption through epithelial or mucocutaneous linings (e.g., oral, rectal, vaginal, and intestinal mucosa, etc.), intradermal, intramuscular, intraperitoneal, intravenous, subcutaneous, infusion, intranasal, epidural, oral, sublingual, intracerebral, intravaginal, transdermal, rectal, by inhalation, or topical, particularly to the ears, nose, eyes, or skin.

Pulmonary administration can also be employed, e.g., by use of an inhaler or nebulizer, and formulation with an aerosolizing agent, or via perfusion in a fluorocarbon or synthetic pulmonary surfactant. For example, the pharmaceutical composition can be formulated as a suppository, with traditional binders and excipients such as triglycerides. Various known delivery systems, including encapsulation in liposomes, microparticles, microcapsules, and capsules, can be used. Thus, the pharmaceutical composition can be delivered in a vesicle, in particular a liposome (see, e.g., Langer (1990) Science 249: 1527-1533; Treat et ai, Liposomes in the Therapy of Infectious Disease and Cancer 317-327 and 353-365 (1989)).

The pharmaceutical composition also can be delivered in a controlled-release system or sustained-release system (see, e.g., Goodson, in Medical Applications of Controlled Release, vol. 2, pp. 1 15-138 (1984)). Other controlled or sustained-release systems previously discussed can be used as well (Langer (1990) Science 249: 1527-1533). For example, a pump can be used (Langer (1990) Science 249: 1527-1533; Sefton (1987) CRC Crit. Ref Biomed. Eng. 14:201; Buchwald et al, (1980) Surgery 88:507; and Saudek et a L, (1989) N. Engl. J Med. 321:574); or polymeric materials can be used (see Langer and Wise (1985) Medical Applications of Controlled Release; CRC Press Inc., U. S.; Smolen and Ball (1984) Controlled Drug Bioavailability, Drug Product Design and Performance; Ranger and Peppas, (1983) J. Macromol. Sci. Rev. Macromol. Chem. 2:61; Levy et ah, (1935) Science 228: 190; During et al, (1989) Ann. Neural. 25:351; and Howard et al, (1989) J. Neurosurg. 71:105). The controlled- or sustained-release systems can be placed in proximity of a target of the compound or a pharmaceutically acceptable salt of the compound, e.g., the respiratory tract, thus requiring only a fraction of the systemic dose.

In embodiments, the EP4 receptor antagonist is provided as a pharmaceutically acceptable salt. The term “pharmaceutically acceptable” can refer to salts or chelating agents are acceptable from a toxicity viewpoint. The term “pharmaceutically acceptable salt” can refer to ammonium salts, alkali metal salts such as potassium and sodium (including mono, di- and tri-sodium) salts for example, alkaline earth metal salts such as calcium and magnesium salts, salts with organic bases such as dicyclohexylamine salts, N-methyl-D-glucamine, and salts with amino acids such as arginine, lysine, and so forth

Other aspects of the invention are directed at methods of treating biofilm production-related disorders in subjects in need thereof. A “biofilm production-related disorder” refers to a disease or disorder that is characterized by a disease-related growth of bacteria in that a biofilm is established. In an embodiment, the disorder affects an epithelial surface, a mucosal surface, or a combination of those surfaces. In particular embodiments of the invention, the surface is a lung surface. In some embodiments, the biofilm production-related disorder is caused by a bacterium.

The method entails administering to the subject an effective amount of an EP4 receptor antagonist that reduces biofilm formation in the subject, and then measuring a reduction or inhibition in the growth of biofilm production-related bacteria in the subject. The reduction in bacterial growth is indicative of the reduction in, or inhibition of, biofilm production in the subject, thereby treating the biofilm production-related disorder. For example, the administered EP4 receptor antagonist can reduce the activity of the EP4 receptor or alter the expression of the EP4 receptor, thereby inhibiting or preventing the formation of a bacterial biofilm.

Non-limiting examples of biofilm production-related disorders include chronic otitis media, prostatitis, cystitis, bronchiectasis, bacterial endocarditis, osteomyelitis, dental caries, periodontal disease, infectious kidney stones, acne, Legionnaire's disease, chronic obstructive pulmonary disease (COPD), and infections from implanted/inserted devices.

In one specific example, subjects with CF display an accumulation of biofilm in the lungs and digestive tract. In subjects afflicted with COPD, such as emphysema and chronic bronchitis, patients display a characteristic inflammation of the airways wherein airflow through such airways, and subsequently out of the lungs, is chronically obstructed. The methods of treatment according to the invention can also benefit a subject having chronic otitis media. Otitis media refers to an infection or inflammation in the middle ear area. The inflammation begins when infections (for example, those caused by bacterial or viral infections) that cause sore throats, colds, or other respiratory/breathing problems spread to the middle ear. Acute otitis media is the presence of fluid, typically pus, in the middle ear with symptoms of pain, redness of the eardrum, and possible fever. However the biofilm production-related disorder can be further classified as chronic if fluid is present in the middle ear for six or more weeks.

Biofilm production-related disorders can also encompass infections derived from implanted/inserted devices (such as those described herein), medical device-related infections, such as infections from biliary stents, surgical and/or orthopedic implant infections, and catheter-related infections (kidney, vascular, peritoneal). An infection can also originate from sites where the integrity of the skin and/or soft tissue has been compromised. Non-limiting examples include dermatitis, ulcers from peripheral vascular disease, a burn injury, and trauma. For example, a Gram-negative bacterium, such as P. aeruginosa, can cause opportunistic infections in such tissues. The ability of P. aeruginosa to infect burn wound sites, e.g., is enhanced due to the breakdown of the skin, burn-related immune defects, and antibiotic selection.

A subject in need of treatment (for example those described herein, such as an animal or human) can be one afflicted with the infections or disorders described above. As such, the subject is at risk of developing a biofilm on or in a biologically relevant surface, or already has developed such a biofilm. Such a subject at risk could be a candidate for treatment with an EP4 receptor antagonist in order to inhibit the development or onset of a biofilm-production-related disorder/condition or prevent the recurrence, onset, or development of one or more symptoms of a biofilm-production-related disorder/condition.

The subject in need can be administered an EP4 receptor antagonist as described herein. It can be administered alone or in combination with a second therapeutic, e.g., such as an additional antibiotic (including these described herein), in order to prevent or inhibit the formation of bacterial biofilms. An antibiotic can be co-administered with the EP4 receptor antagonist, either sequentially or simultaneously.

An antibiotic refers to any compound known to one of ordinary skill in the art that will inhibit the growth of, or kill, bacteria. Useful, non-limiting examples of an antibiotic include lincosamides (clindomycin); chloramphenicols; tetracyclines (such as Tetracycline, Chlortetracycline, Demeclocycline, Methacycline, Doxycycline, Minocycline); aminoglycosides (such as Gentamicin, Tobramycin, Netilmicin, Amikacin, Kanamycin, Streptomycin, Neomycin); beta-lactams (such as penicillins, cephalosporins, Imipenem, Aztreonam); vancomycins; bacitracins; macrolides (erythromycins), amphotericins; sulfonamides (such as Sulfanilamide, Sulfamethoxazole, Sulfacetamide, Sulfadiazine, Sulfisoxazole, Sulfacytine, Sulfadoxine, Mafenide, p-Aminobenzoic Acid, Trimethoprim-Sulfamethoxazole); Methenamin; Nitrofurantoin; Phenazopyridine; trimethoprim; rifampicins; metronidazoles; cefazolins; Lincomycin; Spectinomycin; mupirocins; quinolones (such as Nalidixic Acid, Cinoxacin, Norfloxacin, Ciprofloxacin, Perfloxacin, Ofloxacin, Enoxacin, Fleroxacin, Levofloxacin); novobiocins; polymixins; gramicidins; and antipseudomonals (such as Carbenicillin, Carbenicillin Indanyl, Ticarcillin, Azlocillin, Mezlocillin, Piperacillin) or any salts or variants thereof. Such antibiotics can be obtained commercially, e.g., from Daiichi Sankyo, Inc. (Parsipanny, N.J.), Merck (Whitehouse Station, N.J.), Pfizer (New York, N.Y.), Glaxo Smith Kline (Research Triangle Park, N.C.), Johnson & Johnson (New Brunswick, N.J.), AstraZeneca (Wilmington, Del.), Novartis (East Hanover, N.J.), and Sanofi-Aventis (Bridgewater, N.J.). The antibiotic used will depend on the type of bacterial infection.

Administration of an EP4 receptor antagonist to a subject can serve as a treatment that limits the severity and spread of pathogenic infections, such as bacterial infections. EP4 receptor antagonists intended for human use must be efficacious and function in inhibiting the formation of biofilms, but must also not be toxic. The skilled physician via clinical trials can determine efficacy and toxicity.

An “effective amount” of an EP4 receptor antagonist refers to the amount of a therapy sufficient to reduce or ameliorate the severity and/or duration of a disorder, such as an infection and/or a biofilm production-related disorder. An effective amount of an EP4 receptor antagonist can also be sufficient to reduce the degree and time-span of one or more symptoms associated with an infection and/or biofilm production-related disorder. Additionally, this amount can prevent the advancement of an infection and/or biofilm production-related disorder, cause regression of such a disorder, prevent the recurrence, development, onset or progression of one or more symptoms associated with the infection and/or a biofilm production-related disorder. The skilled physician can determine a therapeutic dose of an EP4 receptor antagonist that inhibits infection and/or biofilm formation and/or reduces the duration of a disorder or symptoms thereof. Methods of administration of an EP4 receptor antagonist composition have been described herein.

An EP4 receptor antagonist according to the methods of the invention can reduce biofilms associated with a biofilm production-related disorder with respect to the surface area the biofilm covers, thickness, and/or consistency (for example, the integrity of the biofilm). This reduction can be assessed via measuring the growth of bacteria associated with biofilm-production-related disorders, conditions, or diseases. For example, the growth of bacteria of a biofilm-production-related disease can be quantified via measuring the density of bacteria of a biofilm-production-related-disease in a biological sample. Non-limiting examples of biological samples include blood, serum, sputum, lacrimal secretions, semen, urine, vaginal secretions, and tissue samples. The reduction in the growth of bacteria of a biofilm-production-related disease can also be measured by chest x-rays or by a pulmonary function test (PFT) (for example, spirometry or forced expiratory volume (FEV)).

In another non-limiting example, the presence or growth of biofilm production-related bacteria can be measured by detecting the presence of antigens of biofilm production-related bacteria in a biological sample, such as those described herein. For example, an antibody to S. aureus components can be used as a test for colonization/infection in a subject afflicted with a biofilm production-related condition or disorder, wherein the presence of Staphylococcus antigens is detected in a biological sample, such as blood. These antibodies can be generated according to methods well established in the art or can be obtained commercially (for example, from Abeam, Cambridge, Mass.; Cell Sciences Canton, Mass.; Novus Biologicals, Littleton, Colo.; or GeneTex, San Antonio, Tex.).

Methods of the invention are provided that can prevent or reduce biofilm formation (such as a bacterial biofilm) on a biologically relevant surface, wherein an EP4 receptor antagonist is administered to a subject (such as a mammal, for example a human) in order to prevent or reduce the formation of bacterial biofilms. These surfaces include, but are not limited to, an epithelial or mucosal surface of the respiratory tract, lungs, the oral cavity, the alimentary and vaginal tracts, in the ear or the surface of the eye, and the urinary tract. For example, a biofilm can affect the surface of a lung or skin, each of which are comprised of epithelial cells.

In embodiments, the EP4 receptor antagonist can be incorporated into a material, such as a bio-compatible material. As used herein, the term “biocompatible” refers to a property of being compatible with a biological tissue or organ without eliciting toxicity, an immune reaction, an injury or the like. In the present invention, the term “biocompatible” in relation to a certain material indicates that when the material is used as it is, the material is biocompatible. Examples of a biocompatible material which may be used in the present invention, include, but are not limited to, PGA, PLA, PCLA, PLGA, poly(L-lactic acid), polybutylate, silicone, biodegradable calcium phosphate, porous 4-fluorinated ethylene resin, polyproplylene, amylose, cellulose, synthetic DNA, polyesters, and the like.

Epithelial cells are named on the basis of their cell type: simple squamous, simple cuboidal, simple columnar, stratified squamous, stratified cuboidal, or stratified columnar epithelia. Such epithelial cells can be obtained from any tissue organ having such cells, for example from the lining of cavities such as the mouth, blood vessels, heart and lungs; from the outer layers of the skin; from the lining of the air passages, stomach, and intestines; in the nose, ears and the taste buds of the tongue; from the lining of the vaginal and urinary tracts, rectum, uterus, and oviducts, and from the larger ducts of certain glands and the papillary ducts of the kidneys. Epithelial cells can also be obtained from in vitro epithelial cell culture systems well known in the art (see, e.g., Harris, A. (ed.), (1996) Epithelial Cell Culture, Cambridge University Press). Such cell lines may be available commercially or can be generated via standard cell culturing techniques (see e.g. Harris, supra).

Other aspects of the current invention are directed to methods that are useful for treating a subject (such as an animal or human) that has, is developing, or is at risk of developing a biofilm-production-related disorder/condition. A subject who is developing a biofilm-production-related disorder/condition is an individual harboring an immature biofilm clinically evident or detectable to the skilled artisan, but that has not yet fully formed. A subject at risk of developing a biofilm can be one in which the introduction of a medical device, a graft implantation, and the like is scheduled. The risk of developing a biofilm can also be due to a biofilm production-related disease (such as the channel transporter mutation associated with CF) that is in its earlier stages, e.g., no bacterial infection and/or biofilm formation is yet detected.

According to the invention, administration of an EP4 receptor antagonist to a subject can serve as a preventive means by which to deter the development of pathogenic infections, such as bacterial infections (eg. S. aureus).

An effective amount of an EP4 receptor antagonist to be administered can be the amount sufficient to prevent the onset or development of a pathogenic infection, such as that associated with a biofilm production-related disease or disorder. The skilled physician can determine a therapeutic dose of an EP4 receptor antagonist that prevents pathogenic infection in addition to biofilm formation. Methods of administration of an EP4 receptor antagonist composition have been described herein.

Aspects of the invention are further directed towards compositions and methods for inhibiting the growth of a microorganism. For example, the method can entail applying an EP4 receptor antagonist to a biofilm and measuring a reduction in the formation of a biofilm. Bacterial biofilms are surface-attached communities of cells that are encased within an extracellular polysaccharide matrix produced by the colonizing cells. Biofilm development occurs via a series of programmed steps, which include an initial attachment to a surface, formation of three-dimensional microcolonies, and the subsequent development of a mature biofilm. Biofilms can be composed of various microorganisms (such as viruses, bacteria, protozoa, and fungi) co-existing within the community and a particular cellular type may predominate. The more deeply a cell is located within a biofilm (such as, the closer the cell is to the solid surface to which the biofilm is attached to, thus being more shielded and protected by the bulk of the biofilm matrix), the more metabolically inactive the cells are. The consequences of this physiologic variation and gradient create a collection of bacterial communities where there is an efficient system established whereby microorganisms have diverse functional traits. A biofilm also is made up of various and diverse non-cellular components and may include, but are not limited to carbohydrates (simple and complex), lipids, proteins (including polypeptides), and lipid complexes of sugars and proteins (lipopolysaccharides and lipoproteins).

Bacterial biofilms exist in nature as well as in medical and industrial environments, such as a GMP facility. The biofilm may allow bacteria to exist in a dormant state for a certain amount of time until suitable growth conditions arise thus offering the microorganism a selective advantage to ensure its survival. However, this selection could pose serious threats to human health in that biofilms have been observed to be involved in about 65% of human bacterial infections (Smith (2005) Adv. Drug Deliv. Rev. 57: 1539-1550; Hall-Stoodley et al., (2004) Nat. Rev. Microbiol. 2: 95-108). In fact, the majority of infections that occur in animals are biofilm-based. In particular, biofilms are problematic with respect to respiratory conditions and diseases.

Harsh treatments (such as chemicals and abrasives) have been used to reduce, prevent, or control biofilm formation. However, biological environments (for example, airways, the urinary tract, wound sites, etc) are particularly sensitive to such harsh treatments. Thus, better methods are needed to control biofilm formation.

In industrial settings, biofilms (comprised of viruses, bacteria, protozoa, fungi, and the like) can adhere to surfaces, such as pipes and filters. Biofilms are problematic in industrial settings because they cause biocorrosion and biofouling in industrial systems, such as heat exchangers, oil pipelines, water systems, filters, and the like (Coetser et al., (2005) Crit. Rev. Micro. 31: 212-32). Thus, biofilms can inhibit fluid flow-through in pipes, clog water and other fluid systems, as well as serve as reservoirs for pathogenic bacteria, protozoa, and fungi. As such, industrial biofilms are an important cause of economic inefficiency in industrial processing systems.

Biofilms (also referred to as “slime residues”) can affect a wide variety of commercial, industrial, and processing operations (such as Good Manufacturing Practices (GMP) facilities). Thus, there is a need for compositions and methods for controlling biofilms in commercial settings as well as biological environments.

The biofilm to be inhibited can be harbored by a subject, can be in vitro, or can be on the surface of an implantable/insertable device to be inserted into a subject. For example, the EP4 receptor antagonist useful in the invention that prevent or reduce the formation of bacterial biofilms can be utilized in order to prevent microorganisms from adhering to surfaces. These surfaces may be hard, semi-hard, porous, soft, semi-soft, regenerating, or. non-regenerating; and can include, but are not limited to, metal, alloy, polyurethane, water, polymeric surfaces of implantable/insertable devices (such as medical devices or catheters), the enamel of teeth, and surfaces of mammalian cellular membranes.

For example, some surfaces can be the surfaces of industrial equipment (such as, equipment located in Good Manufacturing Practice (GMP) facilities, food processing plants, photo processing venues, and the like), the surfaces of plumbing systems, or the surfaces bodies of water (such as lakes, swimming pools, oceans, and the like). The surfaces may be coated, sprayed, or impregnated with an EP4 receptor antagonist prior to use to prevent the formation of bacterial biofilms. Surfaces also may be treated with an EP4 receptor antagonist to reduce, control, or eradicate microorganisms (such as those described above) adhering to such surfaces.

Embodiments can comprise contacting a cell or microorganism with an EP4 receptor antagonist for a period of time sufficient to inhibit the growth of the microorganism, thus preventing or treating a disease or disorder of the invention or preventing or treating biofilm formation. The skilled artisan will recognize that the EP4 receptor antagonist can be contacted with a cell or microorganism for any period of time sufficient to kill the microorganism or prevent or reduce its growth and/or proliferation. In embodiments, the period of time can refer to 1 second, 10 seconds, 30 seconds, 1 minute, 5 minutes, 15 minutes, 30 minutes, 1 hour, or longer than 1 hour.

Application of an EP4 receptor antagonist to a biofilm can be accomplished by any means such as spraying it onto the biofilm, infusing it into the biofilm, pouring it onto the biofilm, wiping it onto the biofilm, or pipetting into the depth of the biofilm, and the like.

Modulation of EP4 receptor antagonist can also result in the reduction or prevention of the formation of a biofilm on semi-solid and solid surfaces. For example, these surfaces can be the surface of implanted and/or inserted devices (a medical device, a catheter, an infusion set of an insulin pump, a stent, a prosthetic graft); a wound dressing; the oral cavity; the alimentary or vaginal tracts; the ears or eyes; a contact lens, in addition to the cases or containers that hold the lenses when not in use; industrial equipment, or plumbing systems.

Additionally, an EP4 receptor antagonist according to the method of the invention can be applied to a surface of a contact lens or an implantable/insertable device and other surgical or medical devices (such as a medical device, a catheter, the infusion set of an insulin pump, a stent, a prosthetic graft, a wound dressing) or a wound site via covering, coating, contacting, associating with, filling, or loading the device with a therapeutic amount of an EP4 receptor antagonist in any known manner including, but not limited to the following: (1) directly affixing to the implant, device, or wound site a therapeutic agent or composition of the EP4 receptor antagonist (for example, by either spraying the implant or device with a polymer/antagonist film, or by dipping the implant or device into a polymer/antagonist solution, or by other covalent or noncovalent means); (2) coating the implant, wound site, or device with a substance, (such as a hydrogel) that will in turn absorb the therapeutic EP4 receptor antagonist composition; (3) interweaving a therapeutic EP4 receptor antagonist composition coated thread (or the polymer itself formed into a thread) into the implant or device or wound site; (4) inserting the implant or device into a sleeve or mesh which is comprised of or coated with a therapeutic EP4 receptor antagonist composition; (5) constructing the implant or device itself with a therapeutic EP4 receptor antagonist composition (or with respect to a wound site, constructing the wound dressing with a therapeutic EP4 receptor antagonist composition; or (6) adapting the implant or device or wound dressing to release the EP4 receptor antagonist composition. Specific disease conditions that are bacteria-based can also benefit from a treatment.

For example, application of an EO4 receptor antagonist onto the surface of implanted and/or inserted devices (as described herein) in order to reduce or prevent bacterial biofilm formation thus allows for long-term implantation and can diminish the resultant likelihood of premature failure of the device due to encrustation and occlusion by such biofilm. The amount of the EP4 receptor antagonist present in a coating, spray, film, and the like (as described herein) applied to the surfaces in order to prevent the formation of a bacterial biofilm is an amount effective to inhibit the attachment of microbes onto the surface and/or the synthesis and/or accumulation of biofilm by attached microbes on such a surface.

Methods of the invention can further protect a subject from premature failure of an insertable or implantable device due to encrustation and occlusion by a bacterial biofilm. According to this method, the subject is administered a therapeutically effective amount of an EP4 receptor antagonist of the invention prior to, at the same time, or after an insertable or implantable device is introduced. The subject is administered the EP4 receptor antagonist that prevents formation of a bacterial biofilm prior to, at the same time, or after the introduction of the implantable/insertable device. Treatment before or after implantation can take place immediately before or after the implantation or several hours before or after implantation, or at a time or times that the skilled physician deems appropriate. According to the present invention, a subject containing a wound site in addition to those subjects receiving implants can harbor a biofilm. For example, an EP4 receptor antagonist can be administered to the subject prior to, during, or after implantation/insertion of a medical device, catheter, stent, prosthesis, and the like or application of a wound dressing. The EP4 receptor antagonist can be administered to the subject according to routes previously described and can further aid in inhibiting biofilm formation on a surface and/or within a subject.

In the case of the oral cavity, the alimentary or vaginal tracts, the ears or eyes, or a contact lens, a therapeutic amount of an EP4 receptor antagonist can be applied via coating, contacting, associating with, filling, or loading the region with a formulation comprising a paste, gel, liquid, powder, tablet, and the like. With respect to the cases or containers that hold the lenses when not in use, industrial equipment, or plumbing systems, an effective amount of an EP4 receptor antagonist can be applied in the same manners as described above. These applications would thus aid in the inhibition of biofilm formation on such surfaces.

In a subject, a biofilm can form on an oral surface (such as teeth, tongue, back of throat, and the like). These biofilms can be associated with day-to-day bacterial activity of natural flora located in such environments, but can also be associated with oral-related disease(s), such as periodontal disease (for example, gingivitis or periodontitis) or dental carries. Application of the EP4 receptor antagonist (according to methods described herein) onto such oral surfaces can inhibit or prevent bacterial biofilm formation. The amount of the EP4 receptor antagonist that can be applied to the surfaces in order to prevent the formation of a bacterial biofilm is an amount effective to inhibit the attachment of microbes onto the surface and/or the synthesis and/or accumulation of biofilm by attached microbes on such a surface.

The EP4 receptor antagonist for use on oral surfaces can comprise a paste formulation (such as toothpaste), which can then be directly applied to the biofilm of such a surface in a subject. The paste formulation can further comprise an abrasive. The EP4 receptor antagonist can also exist as a gel formulation or in liquid formulation. For example, the EP4 receptor antagonist in a liquid formulation (such as a mouthwash) can directly come in contact with the biofilm on the oral surface of a subject

As described herein, aspects of the invention are directed towards compositions and methods of preventing the formation of a biofilm or disrupting a biofilm, such as by contacting a microorganism and/or biofilm with an EP4 receptor antagonist for a period of time sufficient to prevent biofilm formation or disrupt a biofilm. A reduction or inhibition in the growth of biofilm production-related bacteria in the subject can then be determined. For example, this can be tested in a catheter biofilm infection model.

As used herein, the term “inhibitor of biofilm formation,” or “biofilm synthesis inhibitor” (such as an EP4 receptor antagonist) encompasses an agent that inhibits (e.g., disrupts) the attachment of microorganisms onto a surface, to the biofilm matrix itself, to other cells comprising the biofilm, or a combination thereof, and/or inhibits the ability of such microorganisms to produce, synthesize and/or accumulate biofilm on a surface.

Any biofilm-forming organism can comprise the biofilm mass. In certain embodiments, those organisms are one or more viruses, bacteria, protozoa, and fungi. A biofilm can be found on various surfaces and such a surface can be contacted with an EP4 receptor antagonist. In one embodiment, the surface comprises a cellular surface of a subject, an in vitro surface, or an oral surface of a subject. In another embodiment, the surface comprises a cellular surface of a subject, an in vitro surface, or an oral surface of a subject. In particularly useful embodiments, the surface comprises a prosthetic graft, a catheter, a wound dressing, a wound site, a medical device, a contact lens, an implanted device, an oral device, a pipe, or industrial equipment. In further embodiments of the invention, the contacting comprises administering the EP4 receptor antagonist to a subject via subcutaneous, intra-muscular, intra-peritoneal, or intravenous injection; infusion; oral, nasal, or topical delivery; or a combination thereof. In some embodiments, the subject is a human, mouse, rat, bird, dog, cat, cow, horse, or pig. In another embodiment, the EP4 receptor antagonist is applied to the surface of a prosthetic graft to be introduced into a subject. In other embodiments, the EP4 receptor antagonist is applied to the surface of a catheter to be implanted into a subject. In yet further embodiments, the EP4 receptor antagonist is applied to the surface of a wound dressing to be applied on or in a subject. In other embodiments, the EP4 receptor antagonist is applied to the surface of a wound site on a subject. In additional embodiments, the EP4 receptor antagonist is applied to the surface of a medical device to be implanted or inserted into a subject. The subject in many of these instances can harbor the biofilm or has the propensity to form a biofilm. The EP4 receptor antagonist also can be administered to the subject prior to, or during, or after the implantation or insertion of a prosthetic graft, medical device, or a catheter, the application of the wound dressing or to the wound site.

The EP4 receptor antagonist according to the method of the invention can be applied to a surface where a biofilm has formed. In one embodiment, the surface comprises a contact lens, an implanted device, an oral device, a pipe, or industrial equipment. In particular embodiments, industrial equipment is found in a GMP facility. In some embodiments, the industrial equipment comprises a plumbing system. In other embodiments, the surface where a biofilm has formed comprises an oral surface of a subject. In particular embodiments, the biofilm is associated with dental caries while in other embodiments it is associated with periodontal disease. In some embodiments, the EP4 receptor antagonist is in a formulation of a paste, a liquid, a powder, a gel, or a tablet. According to an embodiment of the invention, the EP4 receptor antagonist can be in a paste formulation that can further comprise an abrasive, such as toothpaste. In other embodiments, the EP4 receptor antagonist can be a liquid formulation, such as a mouthwash.

A second therapeutic composition, different than the EP4 receptor antagonist, can also be administered to a subject. In some embodiments of the invention, administration occurs sequentially while in others administration occurs simultaneously. In various embodiments, the therapeutic composition comprises an antibiotic. In yet additional embodiments, the antibiotic comprises a cephalosporin, a macrolide, a penicillin, a quinolone, a sulfonamide, and a tetracycline, or any combination of the listed antibiotics.

Additional aspects of the current invention provide methods for inhibiting biofilm formation on an industrial surface. The method comprises applying an EP4 receptor antagonist to the biofilm on the industrial surface. The EP4 receptor activity or expression of an EP4 receptor on the surface can subsequently be determined. The reduction in the EP4 receptor activity or expression indicates that biofilm formation has been inhibited. In one embodiment, the n EP4 receptor is a bacterial EP4 receptor. Any biofilm-forming organism, such as viruses, bacteria, protozoa, and fungi, can comprise the biofilm. In various embodiments of the invention, the biofilm comprises a viruses, protozoa, fungi, or bacteria.

Inhibition of biofilm formation can be determined by any known method, such as a visual method performed with the aid of a microscope, colorimterically via densitometry, and the like. EP4 receptor antagonists that reduce or prevent the formation of a biofilm on surfaces are described or can be identified via biofilm assays. Thus, one skilled in the art can carry out any known biofilm assay.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1

Prostaglandin E₂ Receptor Antagonist with Antimicrobial Activity Against Methicillin Resistant Staphylococcus aureus

Abstract

Polymicrobial intra-abdominal infections (IAI) involving Candida albicans and Staphylococcus aureus are associated with severe morbidity and mortality (˜80%). Our laboratory discovered that the immunomodulatory eicosanoid, prostaglandin E2 (PGE₂), plays a key role in the lethal inflammatory response during polymicrobial IAI using a mouse model of infection. In studies designed to uncover key PGE2 biosynthesis/signaling components involved in the response, selective eicosanoid enzyme inhibitors and receptor antagonists were selected and pre-screened for antimicrobial activity against C. albicans or S. aureus. Unexpectedly, we found that the EP₄ receptor antagonist, L-161, 982, had direct growth-inhibitory effects on S. aureus in vitro at the physiological concentration required to block PGE2 interaction with EP₄. This antimicrobial activity was observed with methicillin sensitive and methicillin resistant S. aureus (MRSA), with planktonic MIC and MBC values of 50 μg/ml and 100 μg/ml, respectively. In addition, L-161, 982 inhibited S. aureus biofilm formation and had activity against pre-formed mature biofilms. More importantly, treatment of mice with L-161, 982 following i.p. inoculation with a lethal dose of MRSA significantly reduced bioburden and enhanced survival. Furthermore, L-161, 982 protected mice against the synergistic lethality induced by co-infection with C. albicans and S. aureus. The antimicrobial activity of L-161, 982 is independent of EP₄ receptor inhibitory activity; an alternative EP₄ receptor antagonist exerted no antimicrobial or protective effects. Taken together, these findings demonstrate that L-161, 982 has potent antimicrobial activity against MRSA and may represent a significant therapeutic alternative in improving prognosis with mono- or poly-microbial infections involving MRSA.

Introduction

Intra-abdominal infection (IAI) is a broad term used to describe infections that occur as a result of the perforation of the gastrointestinal tract. There is currently a 77% mortality rate associated with polymicrobial IAI involving fungal and bacterial species, a rate that far exceeds that of bacterial monomicrobial infections (20%) (1-4). A plethora of microorganisms can cause IAI, however, two microorganisms that are frequently co-isolated are the fungal pathogen, Candida albicans and the pathogenic bacterium, Staphylococcus aureus (5). In patients with intra-abdominal perforations, isolation of C. albicans alone is indicative of high mortality risk (6). Using animal models, it was demonstrated that co-infection with S. aureus raises the mortality rate even further as a lethal synergistic association exists between these two pathogens (7, 8). Current research is aimed at understanding the mechanism underlying this lethal synergistic interaction as well as host immune response to co-infection.

We recently developed a mouse model of IAI with C. albicans and/or S. aureus and demonstrated that the lethal outcome of co-infection, as opposed to monoinfection, was a result of an amplified host inflammatory response and not dependent on microbial burden. In addition to proinflammatory cytokines, co-infected mice had significantly higher levels of the immunomodulatory prostanoid, Prostaglandin E2 (PGE2) compared to mono-infected mice (8, 9). Strikingly, treatment with the NSAID, indomethacin that reduces PGE2 synthesis, prevented mortality by resolving the pro-inflammatory response demonstrating that the excessive production of PGE2 was a key mediator of the lethal outcome (8). This finding was further supported by the observation that administration of exogenous PGE2 to indomethacin-treated mice restored PGE2 levels, production of pro-inflammatory cytokines, and mortality (8).

PGE2 is derived from polyunsaturated arachidonic acid by the enzymatic action of cyclooxygenases (2 isoforms exist in mammals; COX-1 and COX-2) and can induce either pro- or anti-inflammatory responses (10, 11). PGE2 exerts its biological functions by interacting with one of four specific plasma membrane receptors (designated EP1 through EP4) coupled to guanosine triphosphate (GTP)-binding regulatory proteins (G-proteins) (10). While using selective COX enzyme inhibitors and EP receptor antagonists to identify specific components of the prostanoid biosynthetic and signaling pathways involved in PGE2 production during C. albicans-S. aureus IAI, in vitro antimicrobial activity against C. albicans or S. aureus was assessed. Interestingly, the EP4 receptor antagonist, L-161, 982, exhibited growth inhibitory activity towards S. aureus. The antimicrobial activity of L-161, 982, has not been previously reported; therefore, the goal of these studies was to further investigate the antimicrobial activity of this compound using in vitro assays and in vivo infection models.

Materials and Methods

Strains and growth conditions. The methicillin-resistant S. aureus strain NRS383 used in all experiments was obtained from the Network on Antimicrobial Resistance in S. aureus (NARSA) data bank. NRS383 is positive for the toxic shock syndrome toxin (tst) and 6-toxin genes. SJ-MRSA 6 and SJ-SA5 are methicillin-resistant (MRSA) and methicillin-sensitive (MSSA) clinical S. aureus strains respectively isolated from a patient's catheter. Other bacterial species used in this study include Escherichia coli (ATCC 25922, clinical isolate), S. epidermidis (ATCC 12228), Klebsiella pneumonia (ATCC 43816), Enterococcus faecalis (ATCC 51299, clinical isolate), and Streptococcus mutans (UA159, 12). S. mutans strain was maintained in brain heart infusion (BHI) broth while the other bacterial species were maintained in Luria-Bertani broth. Frozen stocks were obtained at −80° C. and streaked onto agar plates at 37° C. prior to use. A single colony was transferred to 10 ml of culture broth and shaken at 37° C. overnight. On the following day, the overnight culture was diluted 1:100 in fresh growth medium and shaken at 37° C. for 3 h to obtain cells in mid-log growth phase. S. mutans cultures were grown without agitation in the presence of 5% CO2.

The C. albicans strain used in these experiments was DAY185, a prototrophic control strain that has HIS1, URA3, and ARG4 genes reinserted into strain BWP17, an auxotrophic derivative of strain SC5314 (13). Frozen stocks were obtained at −80° C. and streaked onto yeast peptone dextrose (YPD) agar prior to use. A single colony was transferred to 20 ml of YPD broth and shaken at 30° C. for 18 h. Prior to infection, both C. albicans and S. aureus were rinsed 3 times by centrifugation in sterile phosphate-buffered saline (PBS; pH 7.4), counted on a hemocytometer, and diluted in sterile PBS to prepare standardized inocula.

Planktonic Antimicrobial Assay.

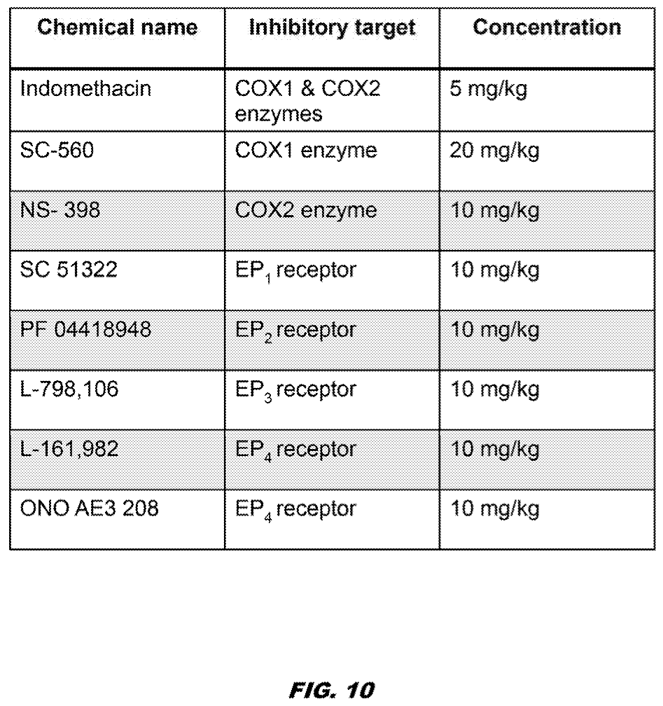

C. albicans and S. aureus were grown as described above. On the following day, cultures were diluted 1:100 in fresh medium containing the following chemical inhibitors at the indicated concentrations: Indomethacin (50 μg/ml, non-selective COX inhibitor, Cayman Chemicals); SC-560 (200 μg/ml, selective COX1 inhibitor, Cayman Chemicals); NS-398 (100 μg/ml, selective COX2 inhibitor, Cayman Chemicals); SC 51322 (100 μg/ml, EP1 receptor antagonist, Tocris); PF 04418948 (100 μg/ml, EP2 receptor antagonist, Tocris); L-798, 106 (100 μg/ml, EP3 receptor antagonist, Tocris); L-161, 982 (100 μg/ml, EP4 receptor antagonist, Tocris); ONO AE3 208 (100 μg/ml, EP4 receptor antagonist, Tocris); and 0.28% dimethyl sulfoxide (DMSO) or 0.28% DMSO alone to serve as controls (FIG. 10). Because the average peritoneal volume of a Swiss Webster mouse is approximately 2 ml, the concentration of each inhibitor and DMSO were chosen to reflect the physiologically delivered final concentrations. Cells were grown with shaking as described above, and 1 ml aliquots were removed each hour in duplicate and optical density at 600 nm measured (OD600) by spectrophotometer (Biomate 3S, Thermo Scientific).

MIC/MBC Assay.

The CLSI reference method for broth dilution antimicrobial susceptibility testing of bacteria was used to determine the MIC of L-161, 982 (CLSI, 2008). Based on inhibition of S. aureus in the planktonic assay, TSB was used for all antimicrobial susceptibility testing of S. aureus. Cells were diluted in sterile TSB to a final concentration of 5.0×105 CFU/ml and 200-μl aliquots (1×105 CFU/well) were added to wells of sterile round-bottom 96-well plates. A stock solution of L-161, 982 was diluted in sterile TSB prior to addition to cells. Drug concentrations tested ranged from 1 to 100 μg/ml and were tested in triplicate. The plates were incubated at 37° C. for 24 h and the OD600 measured using a microplate spectrophotometer (BioTek Instruments, Synergy 2). Bacteriostatic or bactericidal activity was assessed by plating 20 μl of broth from all wells with no visible growth on TSA plates and plates incubated at 37° C. for 24-48 h. The MBC was the lowest drug concentration that prevented growth.

Test for Synergy Between L-161, 982 and Oxacillin.

Due to the newfound antimicrobial activity of L-161, 982 against S. aureus, the checkerboard method (14) with the broth microdilution method, was used to test for synergy with oxacillin, a drug similar to methicillin used more commonly in the clinical setting. Synergy was determined using the fractional inhibitory concentration index (FICI) method (14, 15). With this method, the MIC of the antibiotic compound in combination is divided by the MIC of the compound alone, giving the fractional contribution of each drug component in combination. Quotients for all compounds in combination are summed and drug interactions scored using the formula:

FICI=((MIC AcombA+B)/MICA)+((MIC BcombA+B)/MICB)

The stock solutions and serial twofold dilutions of oxacillin ranged from 8× the MIC to 32× below the MIC and that of L-161, 982 ranged from 8× the MIC to 2000× below the MIC. Oxacillin was serially diluted along the ordinate, while L-161, 982 was diluted along the abscissa. The resulting checkerboard contained each combination of two drugs, with wells that contain the highest concentration of each antibiotic at opposite corners. Each microtiter well was inoculated with 1×105 CFU. The plates were incubated at 37° C. for 24 h and the OD600 measured using a microplate spectrophotometer. A FIC index≤0.5 indicates synergy; a FIC index between 0.5 and 4 indicates indifference; a FIC index>4 indicates antagonism (15, 16).

Biofilm Formation on Polystyrene.

Following growth as described above, microbes were washed in phosphate-buffered saline (PBS) by centrifugation, counted on a hemocytometer, and adjusted to 2×107 CFU/ml in RPMI buffered with MOPS (165 mM). For monomicrobial biofilms, 50 μl of adjusted C. albicans or S. aureus culture was added to each well of a sterile 96-well cell culture polystyrene microtiter plate (1×106 CFU per well); to this, 50 μl of sterile RPMI+MOPS was added. For polymicrobial biofilms, 50 μl of each organism was added per well (1×106 CFU of each organism per well). Plates were incubated for 24 h at 37° C. to induce biofilm formation. Following confirmation of mature biofilm growth by macro- and microscopic examination, plates were washed once with 200 μl sterile saline and then 200 μl RPMI+MOPS containing L-161, 982 at various concentrations (6.25-200 μg/ml) added to each well. L-161, 982-free RPMI+MOPS was added to wells with and without microbes to serve as positive and negative controls, respectively. Plates were incubated at 37° C. for 24 h. After incubation, plates were washed once with PBS and processed for the XTT {sodium 2, 3-bis (2-methoxy-4-nitro-5-sulfophenyl)-5-[(phenylamino) carbonyl]-2H-tetrazolium hydroxide inner salt} metabolic activity assay followed by CFU enumeration.

The XTT reduction assay was used to determine the metabolic activity of biofilms following treatment with L-161, 982 (17). Briefly, wells were washed once in PBS, and plates incubated at 37° C. for 2 h with 200 μl XTT working reagent (0.5 mg/ml XTT and 1 μM Menadione) and the resulting absorbance measured at 490 nm on a microplate spectrophotometer. After L-161, 982 treatment and metabolic activity measurement, enumeration of CFU was carried out as previously described (18).

Mouse Model of Infection.

All animals were housed and handled according to institutionally recommended guidelines. All animal protocols were reviewed and approved by the Institutional Animal Care and Use Committee (IACUC) of the LSU Health Sciences Center New Orleans. Mice were given access to food and water ad libitum. In all experiments, 5-7 week-old female outbred NIH Swiss mice, purchased from Charles Rivers, Frederick, Md. were used.

The mouse peritonitis/intra-abdominal model was performed as previously described (8). In brief, for MRSA peritonitis, mice were injected intraperitoneally (i.p.) with 4×106 CFU or 5×106 CFU or 2×107 CFU of S. aureus in 0.2 ml of PBS containing 3% hog gastric mucin type III (Sigma Aldrich). Mice injected i.p. with 0.2 ml of mucin/PBS served as a negative infection control. For the intra-abdominal infection with C. albicans and S. aureus, mice were injected i.p. with 7×106 CFU of C. albicans and 8×107 CFU of S. aureus (8.7×107 CFU total) in 0.2 ml of PBS. After inoculation, mice were observed over 10 days for signs of morbidity (hunched posture, inactivity, lethargy, and ruffled fur) and mortality. Mice that were significantly moribund were euthanized according to institutionally recommended guidelines. In the MRSA peritonitis experiments, mice were sacrificed at 24 or 48 h post-inoculation to assess microbial burden. Peritoneal cavities were lavaged by injection of 2 ml of sterile PBS followed by gentle massaging of the peritoneal cavity. Peritoneal lavage fluid then was removed using a pipette inserted into a small incision in the abdominal cavity. Spleens were removed, weighed, and mechanically homogenized prior to CFU analysis. Microbial burdens in the peritoneal lavage fluid and spleen were enumerated as described above.

Inhibitor Treatment.

Each inhibitor was prepared fresh as a concentrated stock (20-25 mg/ml) in DMSO and diluted to a working concentration of 10 mg/ml in sterile PBS (final DMSO concentration, 2.3%). A sham treatment containing only 2.3% DMSO in PBS was also prepared. Groups of 5-10 mice were intraperitoneally administered 0.1 ml of inhibitor (as indicated in FIG. 10). For the MRSA peritonitis study, the EP4 receptor antagonist was administered 2 h after S. aureus inoculation and once daily thereafter until day 4 post inoculation. For the IAI co-infection study, COX inhibitor or vehicle control was administered 4 h prior to and 4 and 8 h after inoculation with C. albicans and S. aureus while the EP receptor antagonists were administered daily starting 1 day prior to and 5 days after infection. All other procedures were performed as described above. Control groups received the vehicle at the same dose interval.

Statistical Analysis.

All experiments used groups of 5 to 10 mice and were repeated in duplicate, except where noted. All assays were repeated in triplicate, and the results were averaged. Survival data were analyzed using the Kaplan-Meier test. The metabolic activities of the treated biofilms were compared to the controls using one-way ANOVA with the Tukey post-hoc test. The Mann-Whitney U test was used to analyze all CFU data. In all tests, differences were considered significant at P<0.05. All statistical analyses were performed with Graph Pad Prism software.

Results

L-161, 982 Inhibits Planktonic Growth of S. aureus.

The antimicrobial efficacy of selective COX inhibitors and EP receptor antagonists (FIG. 10) against reference strains, C. albicans DAY185 and S. aureus NRS383 was determined as a prerequisite analysis prior to their use in analyzing the role of prostanoid biosynthetic and signaling pathway during C. albicans-S. aureus IAI. The pharmacological inhibitors have been used in vivo in animal models with no measurable mammalian cell cytotoxicity (19, 20) and thus were tested in vitro in the present study at the relevant physiological concentrations.

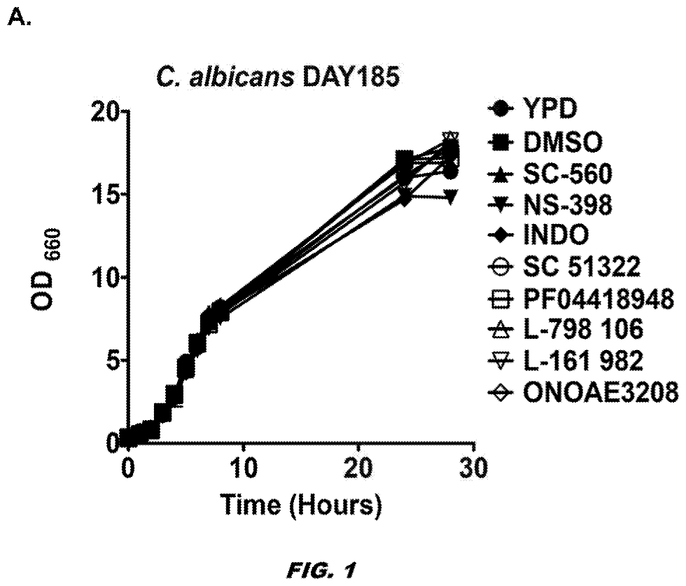

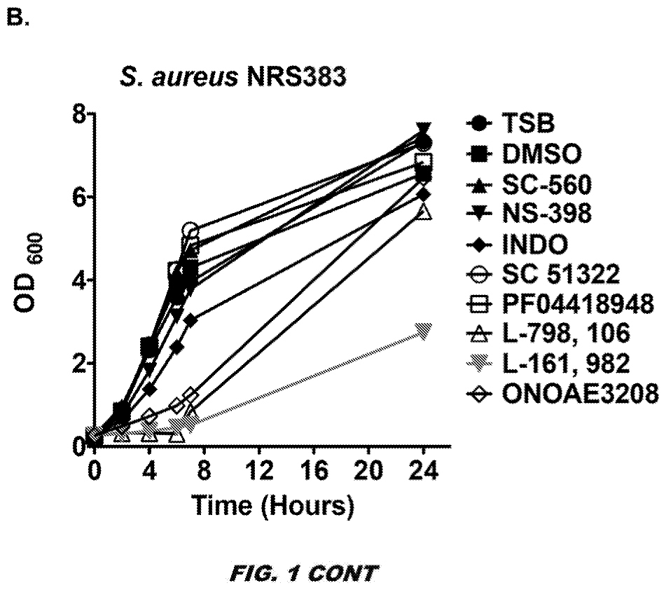

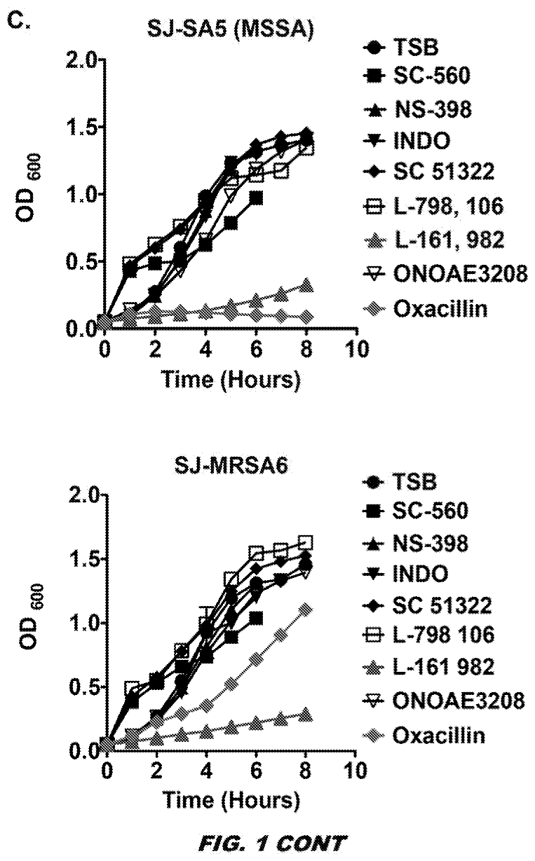

The growth of C. albicans was not inhibited in the presence of COX inhibitors or EP receptor antagonists (FIG. 1, panel A). Similarly, the growth of S. aureus was unaffected by the COX inhibitors as well as EP1-3 receptor antagonists (FIG. 1, panel B). Conversely, the EP4 receptor antagonist, L-161, 982, had significant inhibitory effect on S. aureus growth (FIG. 1, panel B, grey triangle). Based on this significant finding, we extended the antimicrobial susceptibility screen to include clinical MRSA and MSSA strains isolated from a patient's catheter. In all cases, L-161, 982 inhibited growth (FIG. 1, panel C; FIG. 1, panel D). This finding was unexpected because the compound's activity was for blocking signaling of a eukaryotic cell receptor, and was not previously reported to exhibit antimicrobial activity.

Growth Inhibition Kinetics of L-161, 982 on Planktonic Staphylococcal Cells.

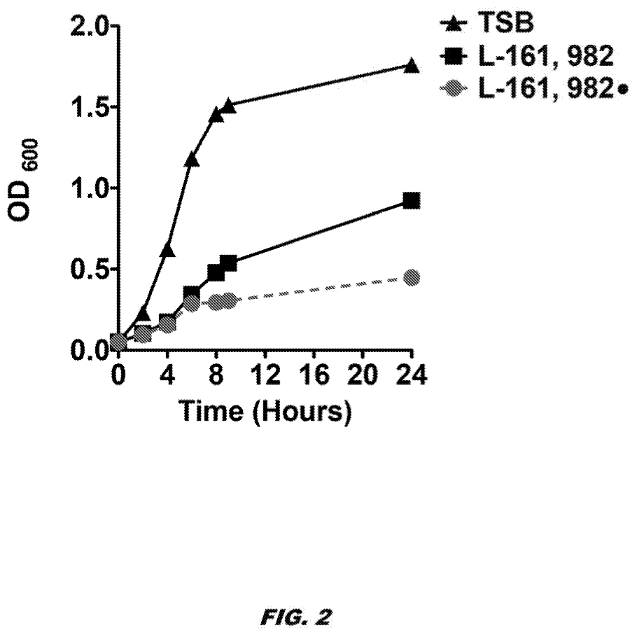

We next investigated the kinetics of growth inhibition kinetics by L-161, 982 against S. aureus and drug stability. The minimum inhibitory concentration (MIC) of L-161, 982 against planktonic S. aureus was 50 μg/ml while the minimum concentration (MBC) was 100 μg/ml. Growth inhibition kinetics of L-161, 982 at the MIC of 50 μg/ml revealed that the inhibitory effect of L-161, 982 on S. aureus was limited to 8 hours (FIG. 2, black square). To address whether the loss of inhibition was due to drug degradation over time (half-life), or to adaptation of S. aureus cells to L-161, 982, fresh L-161, 982 (50 μg/ml) was added during the co-incubation. Results showed that supplementing maintained inhibition over a 24 h period indicating drug degradation or half-life (FIG. 2, grey circle).

L-161, 982 Exerts Anti-Bacterial Activity Against Gram-Positive Bacteria but not Gram-Negative Bacteria.

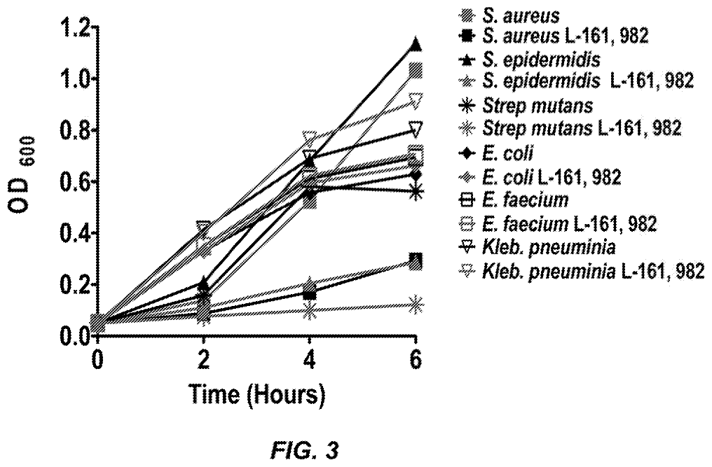

The spectrum of activity of L-161, 982 was investigated by testing several Gram-positive and Gram-negative bacteria. The activity of L-161, 982 was restricted to Gram-positive bacteria that included S. epidermidis and Streptococcus mutans (FIG. 3). No inhibitory effects were observed for the Gram-negative bacteria tested (E. coli, E. faecium, and K. pneumoniae).

L-161, 982 has no potentiating effect on oxacillin. We next tested whether L-161, 982 had synergistic or potentiating effects on antibiotic activity of oxacillin against MRSA using the standard checkerboard broth microdilution assay. The fractional inhibitory concentration (FIC) index of 0.563 indicated indifference (FIC≤0.5=Synergy; FIC 0.5=Indifference; FIC≥4=Antagonism).

L-161, 982 Inhibits S. aureus Biofilm Formation.

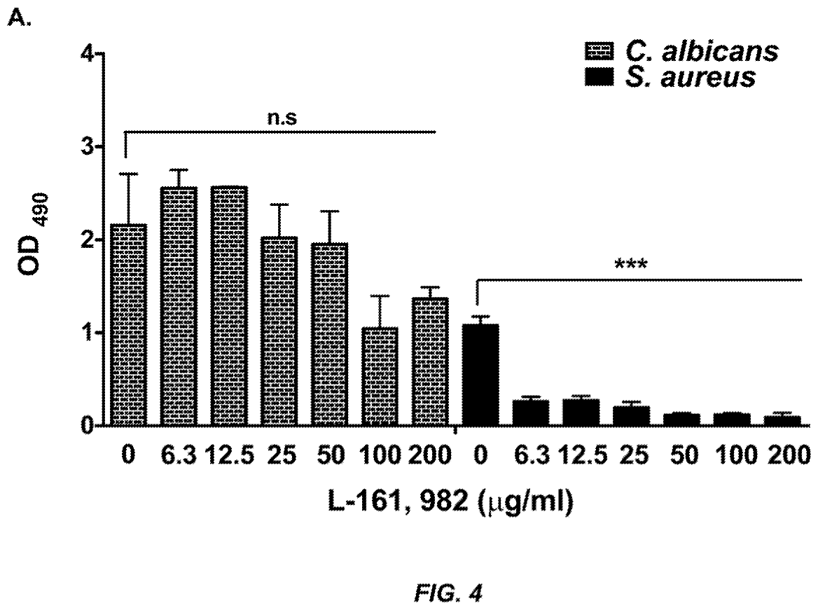

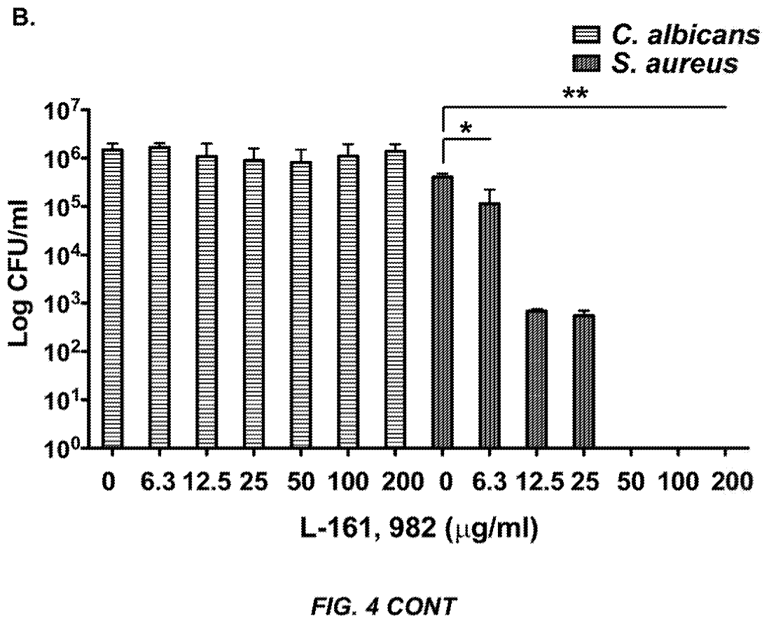

As S. aureus and C. albicans form biofilms that are resistant to most antimicrobials we examined the anti-biofilm potential of L-161, 982 against mono- and dual-species biofilms. For C. albicans biofilm formation no significant inhibition in metabolic activity was observed between treated and untreated mono-species biofilms (FIG. 4, panels A & B). In contrast, metabolic activity of S. aureus was significantly inhibited compared to the untreated control at all concentrations tested (FIG. 4, panel A). In addition, CFUs of S. aureus were significantly reduced by L-161, 982 at concentrations ranging from 6.25 to 25 μg/ml, while no viable cells were recovered at concentrations>50 μg/ml, (FIG. 4, panel B).

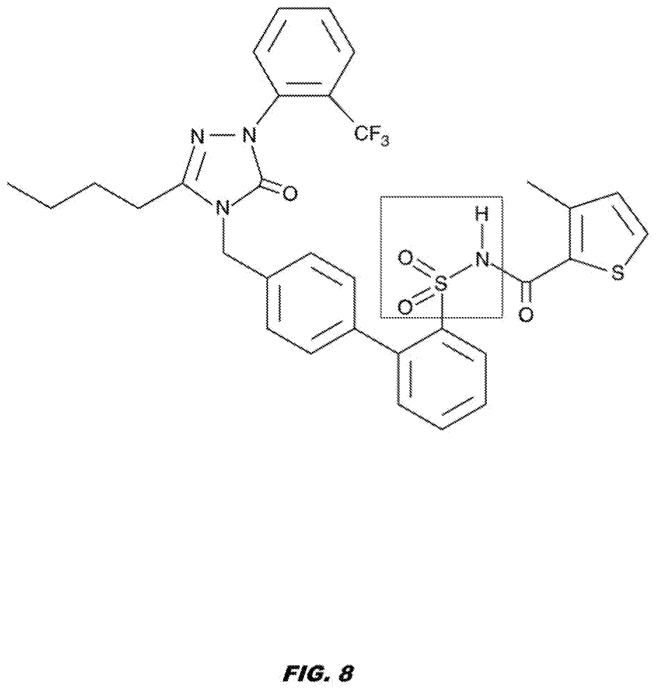

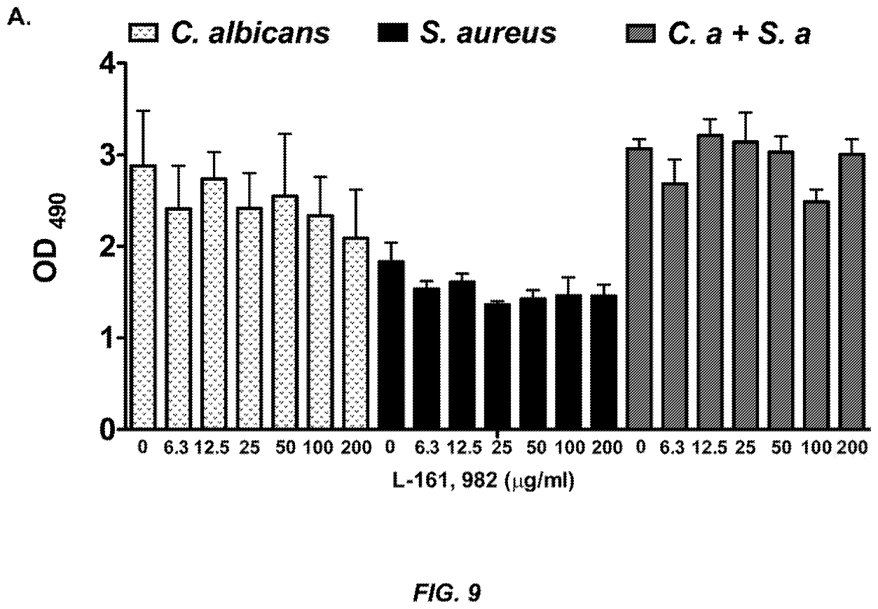

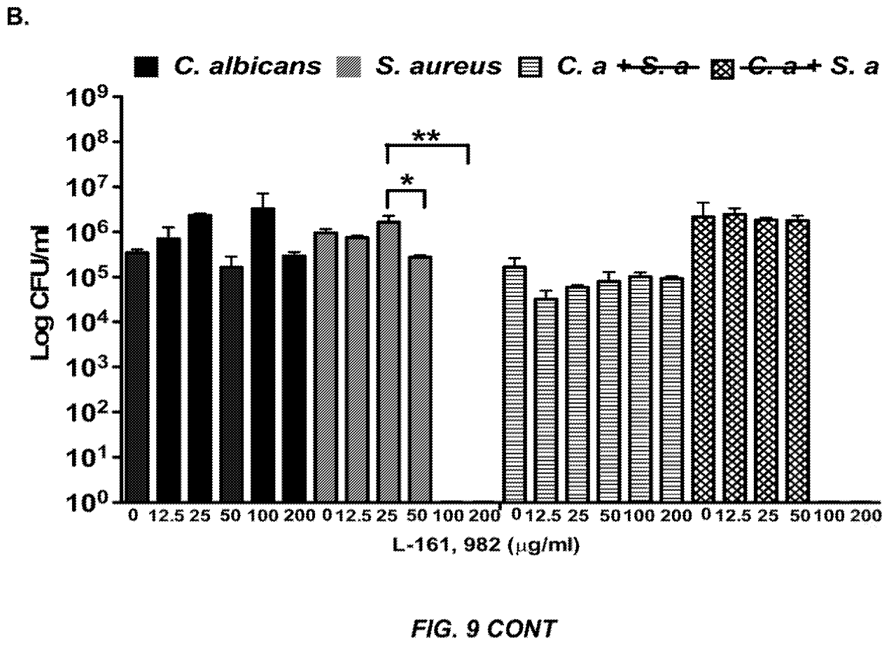

Next we investigated the ability of L-161, 982 to disrupt pre-formed mono- and dual-species biofilms and found no significant inhibitory effects on metabolic activity for both C. albicans and S. aureus at all concentrations tested (FIG. 9). Interestingly though, at the higher concentrations of 100 and 200 μg/ml, no visible biofilm was observed following washing steps and no CFUs were recovered for S. aureus mono- and dual-species biofilm suggesting post-treatment with high concentrations of L-161, 982 may have resulted in weak biofilm structures that were easily disrupted mechanically (FIG. 9).

The Inhibitory of Action of L-161, 982 on S. aureus Growth is not Mediated by Damage to the Cell Membrane or Oxidative Stress.

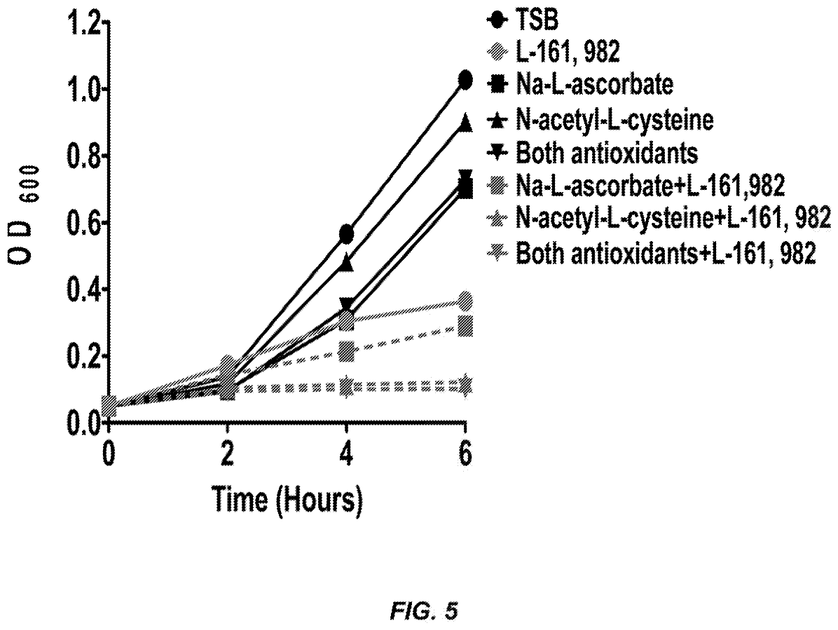

To determine the mechanism of action of L-161, 982 against growth of Gram-positive bacteria, we first looked at its effect on cell membrane integrity using viability staining. Results showed that after 6 h incubation with L-161, 982, S. aureus showed no evidence of cell membrane damaged. A previous study had reported that the inhibitory effect of L-161, 982 on the proliferation of skeletal muscle myoblasts was due to its ability to induce production of high levels of intracellular reactive oxygen species (ROS) that could be reversed by co-treatment with the antioxidants, N-acetyl cysteine or sodium ascorbate (25). Co-treatment with antioxidants did not circumvent the inhibitory effect of L-161, 982 on S. aureus growth (FIG. 5).

L-161, 982 protects against mono- and poly-microbial infections with MRSA. We next evaluated the efficacy of L-161, 982 in vivo using an established mouse peritonitis/sepsis model of infection. Mice were inoculated with three varying lethal inocula of S. aureus and treated with L-161, 982 (10 mg/kg) intraperitoneally (i.p.) 2 h post-inoculation and once daily afterwards through day 4. Control group received the vehicle at the same dose interval.

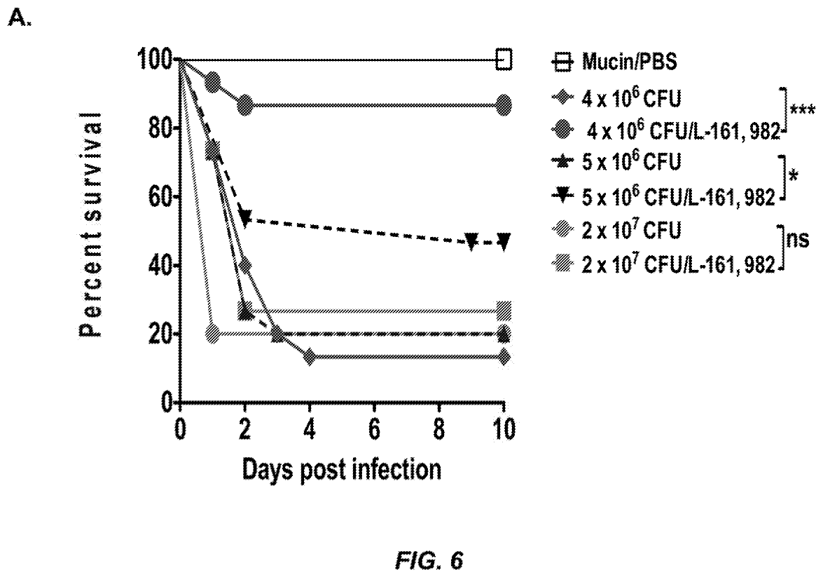

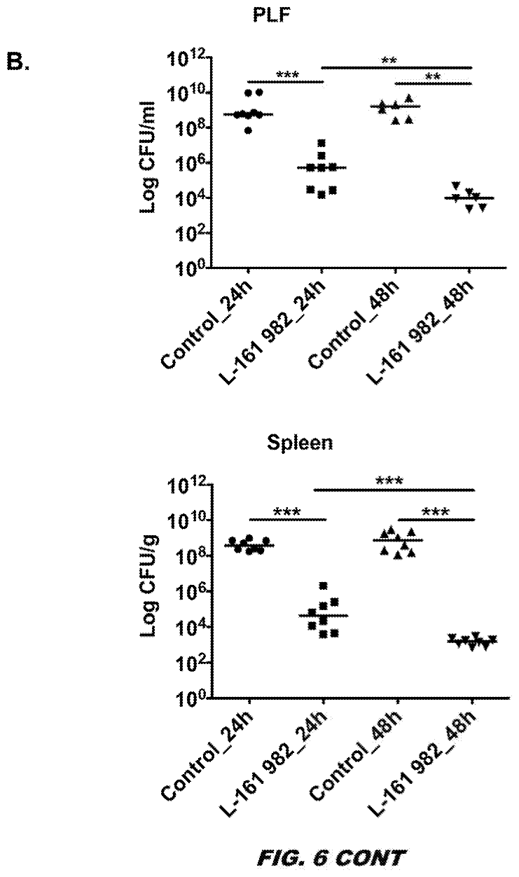

Results showed that at the intermediate inoculum of 5×106 CFU, L-161, 982 increased survival from 25 to 55% by day 2 post-inoculation (FIG. 6, panel A). At the lowest inoculum of 4×106 CFU, survival increased from 40 to 85% during the same 2-day period. Continued dosing of L-161, 982 sustained survival in the intermediate and lowest inocula groups through day 10 compared to 20% survival in the control. At the high inoculum of 2×107 CFU the 80% mortality by day 2 could not be reversed by L-161, 982 treatment. We also investigated the ability of L-161, 982 to aid in bacterial clearance in the host by examining changes in bioburden of S. aureus in the peritoneal cavity and spleen after two or three doses of L-161, 982 (24 h or 48 h post inoculation, respectively). Compared to the untreated group, L-161, 983 significantly reduced S. aureus in the peritoneal cavity and spleen by 3-logs at 24 h and an additional 3-log reduction after a second dose of L-161, 982 (48 h post infection) (FIG. 6, panel B).

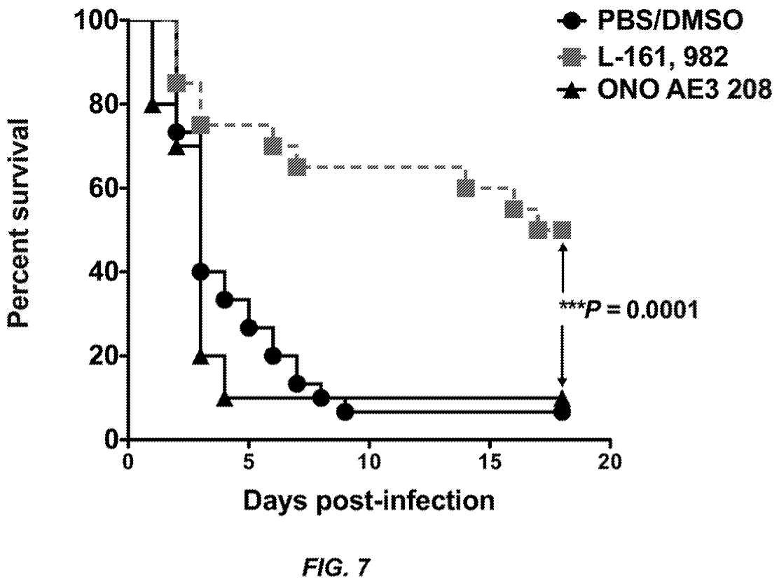

In a final set of experiments, we evaluated the ability of L-161, 982 to reduce lethality in co-infected mice. Mice co-inoculated with the standard 7×106 C. albicans CFU and 8×107 S. aureus CFU (8.7×107 CFU total) were treated with L-161, 982 (10 mg/kg) 2 h post infection and once daily afterwards until day 5. To ensure that the beneficial effect of L-161, 982 was not a result of blocking PGE2-EP4 receptor signaling, we included an alternative antagonist, ONO AE3 208, which was confirmed to have no antimicrobial effect on S. aureus in vitro and in vivo (FIG. 7, FIG. 7). Compared to the vehicle control, mice given L-161, 982 had improved survival from 10 to 50%, while ONO AE3 208 had no effect (FIG. 7).

Discussion

Previous studies from our lab have demonstrated that intra-abdominal infections with C. albicans and S. aureus are associated with a lethal host inflammatory response mediated by the prostanoid, PGE2 (8, 9). In studies designed to uncover the specific pathways involved in PGE2 synthesis and downstream signaling EP receptors using selective COX inhibitors and EP receptor antagonists we discovered that the EP4 receptor antagonist, L-161, 982 had direct antimicrobial activity against S. aureus. This is the first report of a PGE2 receptor antagonist having antimicrobial activity although some NSAIDs have been known to have antimicrobial activity in addition to anti-inflammatory activity (21-24). Therefore, this activity represents a major finding for a PGE2 receptor antagonist that could be exploited as a therapeutic alternative.

It is noteworthy that the S. aureus reference strain used in this study (NRS383) is methicillin-resistant which makes this finding more clinically significant given that MRSA is currently amongst the most common multidrug-resistant pathogens worldwide with urgent need to identify new antibiotics to combat infection. The equivalent activity of L-161, 982 against other clinical MRSA and MSSA isolates obtained from contaminated catheters enhances the potential that L-161, 982 could be a formidable alternative therapeutic against any S. aureus strains.

The MIC for the reference strain is 50 μg/ml while the MBC is 100 μg/ml. Continued dosing of L-161, 982 is required to maintain the growth inhibitory effect, which could either be due to drug degradation (half-life) or depletion. Interestingly, the spectrum of activity of L-161, 982 extends to other Gram-positive bacteria including S. epidermidis and S. mutans, but had no activity against several Gram-negative bacteria. This suggests a narrow spectrum of activity similar to other drug classes specifically targeting Gram-positive bacteria such as β-lactams or macrolides. However, further testing will validate whether this range of activity holds true for all Gram-positive vs. Gram-negative species. We also explored the possible synergistic activity of L-161, 982 with oxacillin and the potential to lower the MIC of oxacillin against MRSA, however, we found the activity of L-161, 982 to be mutually exclusive of oxacillin against MRSA strains.

Blocking the EP4 receptor with L-161, 982 has been reported to inhibit proliferation of mouse primary myoblasts by inducing oxidative stress, an effect that can be circumvented by co-treatment with antioxidants, which reduce intracellular levels of reactive oxygen species (ROS) and restore proliferation (25). Although the prostanoid pathway is not conserved in prokaryotes, we questioned whether L-161, 982 inhibited S. aureus growth by increasing intracellular ROS production. However, growing S. aureus in the presence of L-161, 982 and antioxidants did not reverse the growth inhibitory effect. We also examined the effect of L-161, 982 on membrane integrity by Live/Dead staining and found that membrane disruption is not part of the mechanism of action. Future studies will examine other possible mechanisms of action.

Close examination of the structure of L-161, 982 revealed the presence of the sulfonamide group suggesting L-161, 982 could have the similar antibacterial activity of sulfonamides (FIG. 8). Sulfonamides are bacteriostatic and act as competitive inhibitors of the enzyme dihydropteroate synthase (DHPS), which is involved in folate synthesis (26). One caveat is that sulfonamides are broad spectrum antimicrobial drugs, with activity against both Gram-positive and Gram-negative bacteria. The results of our studies suggest that L-161, 982 is narrow spectrum; therefore, it remains to be determined whether the mechanism of action is attributable to the sulfonamide group. Although extensive toxicological studies have not been carried out, Toxic Dose Low (TDLO) values reported for L-161, 982 subcutaneous administration in mice are 100 μg/kg-333.33 μg/kg (Tocris Bioscience). In our studies using peritoneal administration, L-161, 982 was given at 10 mg/kg body weight based on previous reports in rodents showing that this dose effectively blocked PGE2 interaction with the EP4 receptor with no apparent toxic effects (27, 28). Further studies are required to establish the full range of toxicological properties for L-161, 982.

Both C. albicans and S. aureus are notorious for forming biofilms on medical devices that are extremely resistant to antimicrobial agents and host defense mechanisms, and this becomes more pronounced with mixed-species biofilms. We show here that concentrations of L-161, 982 below the MIC (50 μg/ml) for planktonic growth inhibited S. aureus mono-species biofilm formation, whereas, the bactericidal concentration of 100 μg/ml was required to weaken pre-formed mature biofilms. Unfortunately, L-161, 982 showed no inhibitory effect in a dual-species biofilm with C. albicans, suggesting the presence of C. albicans might have prevented L-161, 982 accessibility. This finding was not surprising as the resulting extracellular matrix (ECM) produced by C. albicans, which is composed of secreted β-1,3-glucan molecules, has been shown to coat bacterial cells within the mixed-species biofilm thereby preventing drug penetration (29). In support of this, the protective nature of C. albicans ECM for S. aureus against vancomycin has been shown whereby no effect on viability was detected in polymicrobial biofilms at the lethal mono-species concentration of 1,600 μg/ml (30, 31). Nevertheless, perhaps the anti-biofilm ability of L-161, 982 can be used against S. aureus or other biofilm forming gut bacteria in a mixed-species biofilm by enhancing the efficacy of other antimicrobials.

Clinically, MRSA biofilms on contaminated medical devices can cause systemic infections and sepsis and are usually associated with high mortality (32, 33). Treatment of MRSA infections depends on the use of combinations of antibiotics such as vancomycin, linezolid, daptomycin, clindamycin, and mupirocin; however, this can lead to development of multi-drug resistant S. aureus strains (34, 35). Our in vivo studies highlight the potential use of L-161, 982 against mono- and poly-microbial infections IAI with MRSA. In a mouse model of MRSA peritonitis, L-161, 982 significantly decreased bioburden in the peritoneal cavity and spleen with just one dose administered 2 h post inoculation. We also found that depending on the infectious dose of S. aureus, one treatment with L-161, 982 raised the survival rate by at least 50%. Consistent with the in vitro results, sustained survival required continued dosing. There was an upper limit to the activity as L-161, 982 was ineffective at a very high inoculum (2×107 CFU) despite continued dosing.

The protective role of L-161, 982 was further demonstrated in a mouse model of polymicrobial intra-abdominal infection with C. albicans where survival was enhanced by 40% (50% survival). Increased survival likely occurred through the antimicrobial effect of L-161, 982 on S. aureus. As discussed earlier, the dose of L-161, 982 used (10 mg/kg) was based on assessing the role of the EP4 receptor in mediating downstream effects of PGE2 signaling during IAI with C. albicans and S. aureus. Coincidentally this same dose had significant antimicrobial activity in vivo. It appears that the antimicrobial activity is independent of the PGE2 signaling aspect as another EP4 receptor antagonist, ONO AE3 208 with no antimicrobial activity, tested in parallel did not enhance survival. Presuming the antagonist effectively blocked PGE2 signaling, it is unlikely that PGE2 promotes lethal inflammation via EP4 in our model. Further studies are aimed at evaluating the pharmacodynamics and pharmacokinetics of L-161, 982 as an antimicrobial agent, as well higher dose responses to determine upper limits of activity.

In summary, this study provides the first evidence of L-161, 982 antibacterial activity via several in vitro designs, and in vivo protection against S. aureus mono- and poly-microbial IAI. In addition, preliminary studies presented here have indicated in vitro activity against two other clinically relevant Gram-positive bacteria. Further studies are required to uncover the growth inhibitory mechanism of L-161, 982 and determine its safety as a therapeutic agent in clinical trials. If successful, L-161, 982 could be used as a preventive or therapeutic alternative agent against mono- and polymicrobial infections of S. aureus including MRSA.

### References Cited in this Examples

1. Blot S I, Vandewoude K H, De Waele J J. 2007. Candida peritonitis. Curr Opin Crit Care 13:195-9.
2. Dupont H, Paugam-Burtz C, Muller-Serieys C, Fierobe L, Chosidow D, Marmuse J P, Mantz J, Desmonts J M. 2002. Predictive factors of mortality due to polymicrobial peritonitis with Candida isolation in peritoneal fluid in critically ill patients. Arch Surg 137:1341-6; discussion 1347.
3. Montravers P, Gauzit R, Muller C, Marmuse J P, Fichelle A, Desmonts J M. 1996. Emergence of antibiotic-resistant bacteria in cases of peritonitis after intra-abdominal surgery affects the efficacy of empirical antimicrobial therapy. Clin Infect Dis 23:486-94.
4. Calandra T, Bille J, Schneider R, Mosimann F, Francioli P. 1989. Clinical significance of Candida isolated from peritoneum in surgical patients. Lancet 2:1437-40.
5. Burke M, Hawley C M, Badve S V, McDonald S P, Brown F G, Boudville N, Wiggins K J, Bannister K M, Johnson D W. 2011. Relapsing and recurrent peritoneal dialysis-associated peritonitis: a multicenter registry study. Am J Kidney Dis 58:429-36.
6. Sandven P, Qvist H, Skovlund E, Giercksky K E. 2002. Significance of Candida recovered from intraoperative specimens in patients with intra-abdominal perforations. Crit Care Med 30:541-7.
7. Carlson E. 1982. Synergistic effect of Candida albicans and Staphylococcus aureus on mouse mortality. Infect Immun 38:921-4.
8. Peters B M, Noverr M C. 2013. Candida albicans-Staphylococcus aureus polymicrobial peritonitis modulates host innate immunity. Infect Immun 81:2178-89.
9. Nash E E, Peters B M, Palmer G E, Fidel P L, Noverr M C. 2014. Morphogenesis is not required for Candida albicans-Staphylococcus aureus intra-abdominal infection-mediated dissemination and lethal sepsis. Infect Immun 82:3426-35.
10. Miller S B. 2006. Prostaglandins in health and disease: an overview. Semin Arthritis Rheum 36:37-49.
11. Ricciotti E, FitzGerald G A. 2011. Prostaglandins and inflammation. Arterioscler Thromb Vasc Biol 31:986-1000.
12. Ajdic D, McShan W M, McLaughlin R E, Savic G, Chang J, Carson M B, Primeaux C, Tian R, Kenton S, Jia H, Lin S, Qian Y, Li S, Zhu H, Najar F, Lai H, White J, Roe B A, Ferretti J J. 2002. Genome sequence of Streptococcus mutans UA159, a cariogenic dental pathogen. Proc Natl Acad Sci USA 99:14434-9.
13. Davis D, Edwards J E, Jr., Mitchell A P, Ibrahim A S. 2000. Candida albicans RIM101 pH response pathway is required for host-pathogen interactions. Infect Immun 68:5953-9.
14. Berenbaum M C. 1978. A method for testing for synergy with any number of agents. J Infect Dis 137:122-30.
15. Berenbaum M C. 1989. What is synergy? Pharmacol Rev 41(2): 93-141.
16. Saiman L. 2007. Clinical utility of synergy testing for multidrug-resistant Pseudomonas aeruginosa isolated from patients with cystic fibrosis: ‘the motion for’. Paediatr Respir Rev 8:249-55.
17. Pierce C G, Uppuluri P, Tummala S, Lopez-Ribot J L. 2010. A 96 well microtiter plate-based method for monitoring formation and antifungal susceptibility testing of Candida albicans biofilms. J Vis Exp doi: 10.3791/2287.
18. Peters B M, Ward R M, Rane H S, Lee S A, Noverr M C. 2013. Efficacy of ethanol against Candida albicans and Staphylococcus aureus polymicrobial biofilms. Antimicrob Agents Chemother 57:74-82.
19. Dey I, Lejeune M, Chadee K. 2006. Prostaglandin E2 receptor distribution and function in the gastrointestinal tract. Br J Pharmacol 149:611-23.
20. Jones V C, Birrell M A, Maher S A, Griffiths M, Grace M, O'Donnell V B, Clark S R, Belvisi M G. 2016. Role of EP₂ and EP₄ receptors in airway micro vascular leak induced by prostaglandin E2. Br J Pharmacol 173:992-1004.
21. de Matos R F, Mendonca L C, da Silva Souza K G, Fonseca A A, Costa E M, de Lima M V, Vieira J M, de Brito M T, Monteiro M C. 2017. Nimesulide inhibits pathogenic fungi: PGE₂-dependent mechanisms. Folia Microbiol (Praha) 62:169-174.
22. Guzman-Beltran S, Rubio-Badillo M A, Juarez E, Hemandez-Sanchez F, Torres M. 2016. Nordihydroguaiaretic acid (NDGA) and alpha-mangostin inhibit the growth of Mycobacterium tuberculosis by inducing autophagy. Int Immunopharmacol 31:149-57.
23. El-Mowafy S A, Abd El Galil K H, El-Messery S M, Shaaban M I. 2014. Aspirin is an efficient inhibitor of quorum sensing, virulence and toxins in Pseudomonas aeruginosa. Microb Pathog 74:25-32.
24. Abdelmegeed E, Shaaban M I. 2013. Cyclooxygenase inhibitors reduce biofilm formation and yeast-hypha conversion of fluconazole resistant Candida albicans. J Microbiol 51:598-604.
25. Mo C, Zhao R, Vallejo J, Igwe O, Bonewald L, Wetmore L, Brotto M. 2015. Prostaglandin E2 promotes proliferation of skeletal muscle myoblasts via EP₄ receptor activation. Cell Cycle 14:1507-16.
26. Henry R J. 1943. The mode of action of sulfonamides. Bacteriol Rev 7:175-262.
27. Machwate M, Harada S, Leu C T, Seedor G, Labelle M, Gallant M, Hutchins S, Lachance N, Sawyer N, Slipetz D, Metters K M, Rodan S B, Young R, Rodan G A. 2001. Prostaglandin receptor EP₄ mediates the bone anabolic effects of PGE₂. Mol Pharmacol 60:36-41.
28. Greaves E, Home A W, Jerina H, Mikolajczak M, Hilferty L, Mitchell R, Fleetwood-Walker S M, Saunders P T. 2017. EP₂ receptor antagonism reduces peripheral and central hyperalgesia in a preclinical mouse model of endometriosis. Sci Rep 7:44169.
29. Kong E F, Tsui C, Kucharikova S, Andes D, Van Dijck P, Jabra-Rizk M A. 2016. Commensal protection of Staphylococcus aureus against antimicrobials by Candida albicans biofilm matrix. MBio 7(5). pii: e01365-16. doi: 10.1128/mBio.01365-16.
30. Harriott M M, Noverr M C. 2009. Candida albicans and Staphylococcus aureus form polymicrobial biofilms: effects on antimicrobial resistance. Antimicrob Agents Chemother 53:3914-22.
31. Harriott M M, Noverr M C. 2010. Ability of Candida albicans mutants to induce Staphylococcus aureus vancomycin resistance during polymicrobial biofilm formation. Antimicrob Agents Chemother 54:3746-55.
32. Jean S S, Hsueh P R. 2011. Current review of antimicrobial treatment of nosocomial pneumonia caused by multidrug-resistant pathogens. Expert Opin Pharmacother 12:2145-8.
33. Hall M J, Williams S N, DeFrances C J, Golosinskiy A. 2011. Inpatient care for septicemia or sepsis: a challenge for patients and hospitals. NCHS Data Brief: (62): 1-8.
34. Martin G S. 2012. Sepsis, severe sepsis and septic shock: changes in incidence, pathogens and outcomes. Expert Rev Anti Infect Ther 10:701-6.
35. Liu C, Bayer A, Cosgrove S E, Daum R S, Fridkin S K, Gorwitz R J, Kaplan S L, Karchmer A W, Levine D P, Murray B E, M J R, Talan D A, Chambers H F. 2011. Clinical practice guidelines by the infectious diseases society of America for the treatment of methicillin-resistant Staphylococcus aureus infections in adults and children: executive summary. Clin Infect Dis 52:285-92.

### Example 2

### Antimicrobial Activity of EP4 Receptor Antagonist L 161,982

We have discovered a new use for the compound L-161,982, CAS No. 147776-06-5. This compound is used as a human receptor antagonist. Biological activity: selective EP4 receptor antagonist; EP4=prostaglandin E2 receptor 4. Prostaglandin E2 (PGE2) is a well-known host immune system signaling lipid that acts on target cells via specific receptors (EP1-4). We tested several of these receptor antagonists for antimicrobial activity in vitro, and found that L-161,982 inhibits the growth of Staphylococcus aureus and other gram positive species such as S. epidermidis and Streptococcus mutans. Further experiments demonstrated that L-161,982 reduced S. aureus burden in vivo, using an animal model of peritonitis, and also improved survival. Commercial applications would include use as an antimicrobial agent, delivered either topically or systemically. In addition, it could be incorporated into medical device polymers or used as a coating agent, to prevent contamination or formation of bacterial biofilms in the clinical setting. Without wishing to be bound by theory, it will have acceptable levels of toxicity in vivo because it is routinely used on cultured mammalian cells as a receptor antagonist.

There is no documented use for L-161,982 as an antimicrobial drug, nor has any activity been documented for this compound for prokaryotic organisms. The only documented use is as a mammalian receptor antagonist.

For clinical use, this would require drug toxicity testing, and pharmacokinetics and pharmacodynamics studies to determine drug half-life, drug metabolism, methods of dosing, route of delivery, potential adverse events.

### Example 3

Mechanism of Action of a Prostaglandin E2 Receptor Antagonist with Antimicrobial Activity Against Staphylococcus aureus

Our laboratory recently reported that the EP4 receptor antagonist, L-161, 982, had direct growth-inhibitory effects on S. aureus in vitro and in vivo, reducing microbial burden and providing significant protection against lethality in models of S. aureus monomicrobial and polymicrobial intra-abdominal infection. This antimicrobial activity was observed with both methicillin-sensitive and methicillin-resistant S. aureus (MRSA), as well as other Gram-positive bacteria. The antimicrobial activity of L-161, 982 was independent of EP4 receptor inhibitory activity. In this study, we investigated the mechanism of action (MOA) of L-161, 982, which contains a sulfonamide functional group. However, results demonstrate L-161, 982 does not affect folate synthesis (sulfonamide MOA), oxidative stress, or membrane permeability. Instead, our results suggest that the inhibitor works via effects on inhibition of the electron transport chain (ETC). Similar to other ETC inhibitors, L-161, 982 exposure results in a small colony size variant phenotype and inhibition of pigmentation, as well as significantly reduced hemolytic activity, and ATP production. Taken together, these findings demonstrate that L-161,982 has potent antimicrobial activity against MRSA via inhibition the electron transport chain, representing a new member of a potentially novel antimicrobial drug class.

Introduction

Staphylococcus aureus is a clinically significant gram positive bacterial pathogen, causing a wide range of nosocomial infections ranging from pneumonia and skin or surgical site infections, to systemic bloodstream infections and sepsis. S. aureus is the leading cause of infection in critically ill and injury patients (1). There has been an alarming increase in methicillin-resistant S. aureus strains (MRSA) and multi-drug resistant (MDR) strains. These resistant strains are prevalent in both hospital-associated and community-acquired infections, which are associated with prolonged hospitalization and treatment costs and mortality rates of 20-50% (2, 3). Is it estimated that >200,000 MRSA infections occur per year, costing approximately $3.3 billion (4).

MRSA also causes polymicrobial infections, including systemic infections with the fungal pathogen Candida albicans (5). Using animal models, it was demonstrated that polymicrobial infection with C. albicans and S. aureus causes synergist effects on mortality compared with monomicrobial infections (6-8). Our laboratory discovered that the immunomodulatory eicosanoid, prostaglandin E2 (PGE2), plays a key role in the lethal inflammatory response during polymicrobial intra-abdominal infection (IAI) using a mouse model of infection (6, 9). In studies designed to uncover key PGE2 biosynthesis/signaling components involved in the response, selective eicosanoid enzyme inhibitors and receptor antagonists were selected and pre-screened for antimicrobial activity against C. albicans or S. aureus. Unexpectedly, we found that the EP4 receptor antagonist, L-161, 982, had direct growth-inhibitory effects on S. aureus in vitro at the physiological concentration required to block PGE2 interaction with EP4 (10). This antimicrobial activity was observed with both methicillin-sensitive and methicillin-resistant S. aureus (MRSA), with planktonic MIC and MBC values of 50 μg/ml and 100 μg/ml, respectively. In addition, L-161, 982 inhibited S. aureus biofilm formation and had activity against pre-formed mature biofilms.

In vivo testing demonstrated that treatment of mice with L-161, 982 following intra-peritoneal inoculation with a lethal dose of MRSA significantly reduced microbial burden and enhanced survival (9). Furthermore, L-161, 982 protected mice against the synergistic lethality induced by co-infection with C. albicans and S. aureus. Importantly, administration of L-161, 982 alone did not cause any overt signs of toxicity, with no morbidity or mortality observed in control animals. An alternative EP4 receptor antagonist exerted no antimicrobial or protective effects, indicating that the antimicrobial activity of L-161, 982 is independent of its effects on inhibiting EP4 receptor activity. The goal of these studies was to further investigate the antimicrobial mechanism of action (MOA) of L-161, 982, testing mechanisms associated with major classes of antimicrobials.

Materials and Methods

Strains and growth conditions. The methicillin-resistant S. aureus strain NRS383 was obtained from the Network on Antimicrobial Resistance in S. aureus (NARSA) data bank. As NRS383 has little or no hemolytic activity or pigmentation, S. aureus strain 43300 (ATCC) was used for the hemolysis and staphyloxanthin assays. Frozen stocks were obtained at −80° C. and streaked onto trypticase soy agar (TSA) plates at 37° C. prior to use. A single colony was transferred to 10 ml of trypticase soy broth (TSB) and shaken at 37° C. overnight. On the following day, the overnight culture was diluted 1:100 in fresh growth medium and shaken at 37° C. for 3 h to obtain cells in mid-log growth phase.

Effect of exogenous thymidine on antibacterial activity of L-161, 982. S. aureus was cultured to mid-log phase in cation-adjusted Mueller-Hinton II broth with shaking at 37° C. Cultures were further diluted to yield a final concentration of 5×105 cells/ml. and treated with drugs alone or in combination with 200 μg/ml of thymidine (Sigma). Drugs were L-161, 982 (50 μg/ml) or 40 μg/ml of trimethoprim-sulfamethoxazole (SXT) at a ratio of 1:19 (2 μg/ml of trimethoprim and 38 μg/ml of sulfamethoxazole; Sigma) as a positive control. Cultures were incubated at 37° C. without shaking and aliquots were removed at t(0), 2, 4, 6, and 24 hours. CFUs were enumerated by plating various dilutions on TSA plates and incubating at 37° C. for 24 hours. Results were normalized to negative control cultures containing no drug at each time point.

Cytoplasmic leakage assay. Cytoplasmic leakage assays were performed as previously described with modifications (11). Briefly, S. aureus was cultured to mid-log phase in TSB with shaking at 37° C. Bacteria were then harvested and resuspended in PBS to a concentration of ˜108/ml. Cultures were treated with L-161,982 (50 μg/ml) or Chlorhexidine (4 μg/ml), a positive control, and incubated at 37° C. with shaking. Culture supernatants were monitored over time for cytoplasmic leakage via OD260 measurements using a spectrophotometer (Biomate 3S, Thermo Scientific). For this, aliquots were removed at 0.5, 1, 2, and 3 hours and centrifuged for 5 min at 10,000×g at room temperature to remove cellular debris before taking OD260 measurements. Negative control cultures containing no drug were used to calculate change in absorbance (AA260) at each time point.

Generation of hydroxyl radicals. Antibiotic induced hydroxyl radical production was measured as previously described (12). Overnight cultures of S. aureus were diluted 1:100 in TSB and grown for 3 h at 37° C. with shaking to mid-log phase. Cultures were diluted in TSB to OD600=0.05 and L-161, 982 was added at a final concentration of 50 □g/ml+/−H2O2 (1 mM; Sigma) or thiourea (150 mM; Sigma). Cultures were incubated at 37° C. with shaking and aliquots were removed at t(0) and at 1 h intervals duplicate, diluted 1:100 and optical density measured at 600 nm (OD600) or serially diluted and plated onto SDA and grown at 37° C. for 24 h for CFU enumeration.

Hemolysis Assay. Hemolytic activity was measured using Mueller-Hinton agar plates containing 5% defribinated sheep blood (Thermo Fisher)+/−L-161, 982 at a final concentration of 50 μg/ml. To make uniform lawns, S. aureus was spread in 1 cm spots and plates were incubated at 37° C. for 24 h followed by 24 h incubation at 4° C. Plates were placed on a light box and digitally photographed and images were analyzed using Photoshop CS3. The zone of red blood cell clearance was defined by selecting the area of clearance proximal to the area of growth using the color selection tool set to 25% threshold. The width of each zone was measured at 0, 90, 180, 270° around growth spots (2-3 spots/group) using the ruler tool. Measurements were normalized to petri plate gridlines (13 mm) for each plate.

Staphyloxanthin Assay. S. aureus was diluted 100-fold in 50 ml of TSB containing 50 or 100 μg/ml of L-161,982 and cultured shaking at 37° C. for 48 h. Control cultures were grown in broth alone. Cells were harvested by centrifugation (4000 rpm) and washed twice with PBS. Cells from each culture were counted and normalized. Normalized cell suspensions were centrifuged (4000 rpm), resuspended in 1 ml of methanol and incubated at 55° C. for 30 minutes to extract pigment. Suspensions were centrifuged at maximum speed (14000 rpm) to remove cell debris, leaving the supernatant containing pigment. Optical densities of supernatants were measured at 465 nm using a spectrophotometer.

ATP detection assay. S. aureus was diluted 100-fold in 10 ml of fresh Mueller Hinton II broth which was used as the inoculum. The inoculum was incubated with various concentrations of L-161,982 (12.5-200 μg/ml) in a 96-well plate in triplicate for 5 h at 37° C. After incubation, cells L-161,982 were stained with LIVE/DEAD Baclight bacterial viability stain (Invitrogen) and viable cells were counted. Samples were normalized to viable cell number before transferring to an opaque-walled 96-well plate. An equal volume of BacTiter-Glo™ Reagent (Promega) was added. The plate was allowed to develop for 5 min at room temperature. Luminescence was quantified using a BioTek Synergy 2 plate reader. A media alone control was used to detect background luminescence.

Statistical Analysis. In all assays, samples were analyzed in duplicate and independently repeated. Statistical differences among groups were analyzed using one-way ANOVA and comparisons of each experimental group with control groups were analyzed using the Student's unpaired t test (2 tailed, unequal variance). In all tests, differences were considered significant at P<0.05. All statistical analyses were performed with Graph Pad Prism software.

Synergy Studies—Motyl M, Dorso K, Barret J, Giacobbe R. Basic microbiological techniques for antibacterial drug discovery. Curr Protoc Pharmacol. 2005; 13A.3.1-13A.3.22., which is incorporated by reference herein in its entirety.

Results

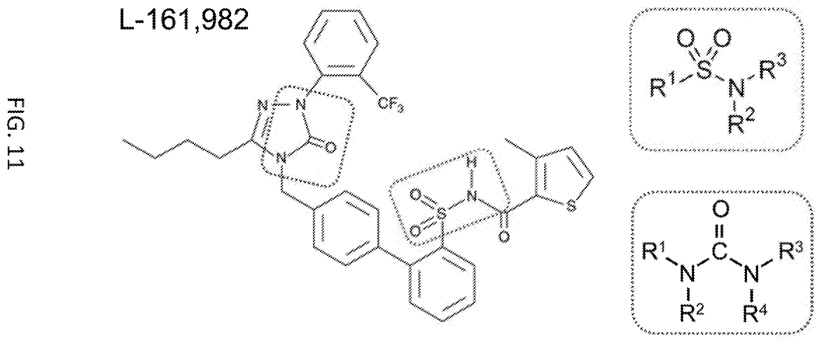

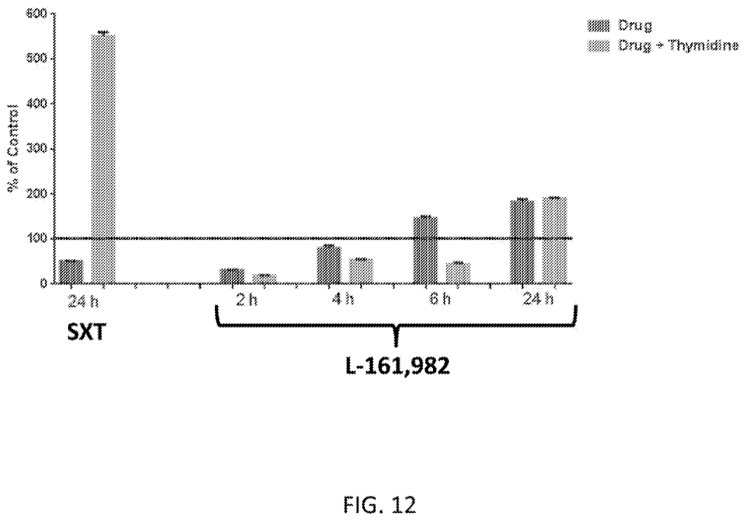

Growth inhibitory effect of L-161, 982 is not due to inhibition of folate synthesis. Examination of the chemical structure of L-161, 982 revealed the existence of several functional groups found in different classes of antibiotics. Notably, it has a sulfonamide functional group (—S(═O)2-NR2), which consists a sulfonyl group connected to an amine group (FIG. 11), similar to the first commercial synthetic antibiotics (sulfonamides or sulfa drugs). The sulfonamide group is located within L-161,982, resulting in a secondary amine group, similar to the sulfonamide antibiotic sulfamethoxazole. The inhibitory mechanism of sulfonamides on bacterial growth is mediated by inhibition of dihydrofolate reductase, a key enzyme involved in folate synthesis (13). The inhibitory effects of sulfonamides can be antagonized by extracellular thymidine, which provides bacteria an alternative means to folate synthesis (14, 15). However, in contrast to SXT (positive control drug), addition of exogenous folate did not reverse the growth inhibition exerted by L-161, 982 S. aureus growth, suggesting the mechanism of action of this drug is unrelated to the sulfonamide group (FIG. 12).

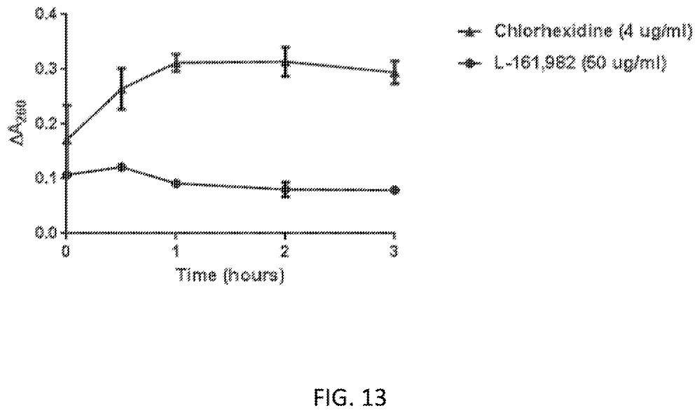

The inhibitory of action of L-161, 982 on S. aureus growth is not mediated by damage to cell membrane or bacteriolysis. Another major target of several classes of antibiotics is the bacterial cell membrane. To monitor membrane damage, the release of nucleic acids and other intracellular 260 nm absorbing material was measured in culture supernatants. In contrast to the results for chlorhexidine (positive control drug), The OD260 values of culture supernatants from cultures treated with L-161, 982 did not increase over the 24 h treatment period (FIG. 13). These finding strongly indicate no loss of bacterial cell membrane integrity occurs following L-161, 982 treatment.

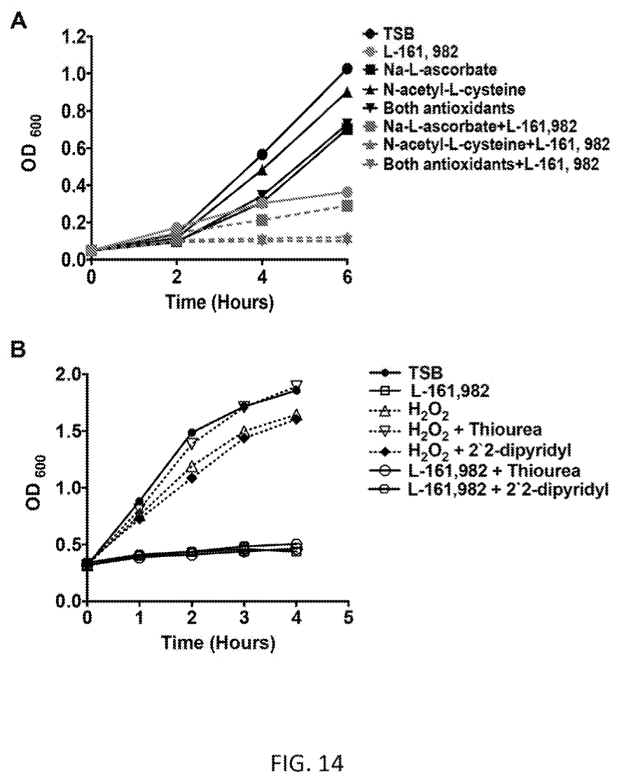

L-161, 982 does not induce oxidative stress. A previous study had reported that the inhibitory effect of L-161, 982 on the proliferation of skeletal muscle myoblasts was due to its ability to induce production of high levels of intracellular reactive oxygen species (ROS) that could be rescued by co-treatment with the antioxidants, N-acetyl cysteine or sodium ascorbate (16). Co-treatment with these antioxidants did not circumvent the inhibitory effect of L-161, 982 on S. aureus growth (FIG. 14, panel A). Using a potent hydroxyl radical scavenger, thiourea, we further investigated the possibility that L-161, 982 induced oxidative stress by generating hydroxyl radicals in S. aureus. Again, we found that in the presence of L-161, 982, thiourea did not alleviate its inhibitory effect on S. aureus growth suggesting the drug did not trigger the generation of hydroxyl radicals (FIG. 14, panel B).

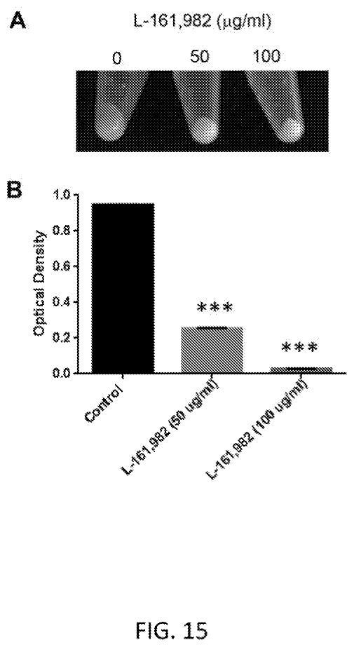

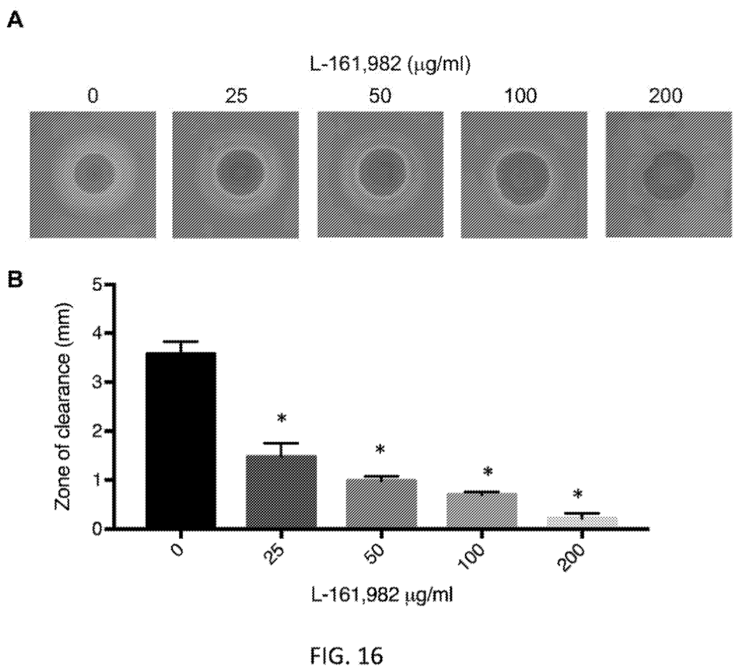

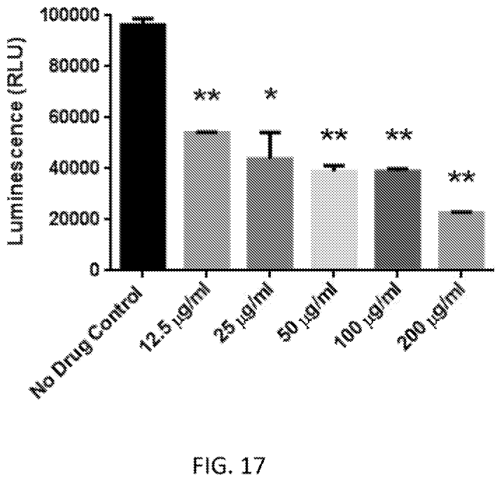

L-161, 982 inhibits activities associated with the electron transport chain including ATP production. In addition to the sulfonamide group, the chemical structure of L 161, 982 contains a urea group connected to two aryl groups (diarylurea) (FIG. 11). Urea derivatives are commonly used as herbicides, interfering with electron transport (17). Diarylurea compounds have been shown to have similar inhibitory effects on S. aureus electron transport, resulting in distinct phenotypes including small colony variant (SCV), inhibition of pigment production, and reduced hemolytic activity (18). SCVs typically lack a functional electron transport chain and cannot produce virulence factors such as hemolysins or the antioxidant pigment staphyloxanthin [reviewed in (19)]. In the presence of L-161, 982, S. aureus also grew as small colonies on agar plates and resulted in loss of yellow pigmentation (staphyloxanthin) (FIG. 15, panels A&B). Addition of L-161, 982 to blood agar resulted in significantly reduced zones of clearance, indicative of reduced hemolysin production (FIG. 16, panel A). These defects are all indicative of interruption of electron transport as carotenoid formation as well as toxin production is dependent on an active electron transport (18, 20, 21). During aerobic respiration, the force required for driving ATP synthesis requires a transmembrane electrical potential (membrane potential), which is generated by the electron transport chain. Addition of L-161,982 to S. aureus significantly inhibited ATP production (FIG. 17). Collectively, these findings suggest that the mechanism of action of L-161, 982 on S. aureus is via inhibition of the electron transport chain.

Discussion

Antibiotic classification is based upon mechanism of action and drug-target interaction. The three major targets for antibiotics include cell wall/membrane synthesis/integrity, nucleic acid synthesis, and protein synthesis. We recently published that the mammalian EP4 receptor antagonist, L-161, 982, had direct growth-inhibitory effects on S. aureus in vitro at the physiological concentration required to block PGE2 interaction with EP4 (10). The antimicrobial activity of L-161, 982 was not related to inhibitory effects on host EP4 receptor. Therefore, it was unlikely that the mechanism of action of growth inhibition was related to inhibition of any potential receptor homologs in S. aureus.

In eukaryotes, L-161, 982 has also been reported to inhibit proliferation of mouse primary myoblasts by inducing oxidative stress, an effect that can be circumvented by co-treatment with antioxidants, which reduce intracellular levels of reactive oxygen species (ROS) and restore proliferation (16). In addition, a common mechanism of action of several classes of antibacterial drugs is via induction of hydroxyl radical formation in both Gram-negative and Gram-positive species, which can be ablated by addition of hydroxyl radical scavengers (12). However, addition of either antioxidants or hydroxyl radical scavengers reversed the growth inhibitory effect of L-161, 982 on S. aureus.

The chemical structure of L-161, 982 contains several functional groups, including a sulfonamide group, that could be responsible for its antibacterial activity. Sulfonamides are bacteriostatic antibiotics that act as competitive inhibitors of the enzyme dihydropteroate synthase (DHPS) involved in folate synthesis, which leads to defective thymidine production and subsequent DNA synthesis (22). Addition of exogenous thymidine rescues growth inhibition in the presence of folate synthesis inhibitors (15). However, our findings demonstrate that exogenous thymidine had no effect on the antimicrobial activity of L-161, 982 against S. aureus arguing against folate synthesis inhibition as the drug's mechanism of action. Disruption of membrane integrity, another major mechanism of action of antibacterial drugs, was also unlikely as L-161, 982 had no effect on bacteriolysis or membrane damage.

In S. aureus, it was previously reported that several diaryl urea compounds exert antibacterial activity via inhibition of the electron transport chain (18). L-161, 982 also contains a urea group flanked by two aryl groups, and may have similar effects on S. aureus. The electron transport chain enables aerobic respiration and subsequent ATP production and is a less common target for antimicrobial agents. Distinct phenotypic characteristics associated with interruptions in electron transport include slow growth as cell wall biosynthesis is dependent on copious amounts of ATP, decreased pigmentation as carotenoid formation (staphyloxanthin) requires electron transport, and production of hemolysins (18, 23). Our initial observation that L-161, 982 slowed growth of S. aureus suggested a potential effect on the electron transport chain (10). In agreement with this observation, exposure to L-161, 982 also resulted in small colony variant (SCV) phenotype similar to diaryl urea compounds (18). Subsequent testing revealed that L-161, 982 inhibited pigment production and hemolytic activity, also consistent with inhibition of the electron transport chain. Finally, L-161, 982 inhibited ATP as the end product of the electron transport chain, which served as the most direct evidence for its mode of action. It is also possible that ETC inhibitors may exert synergy with other drugs that target ATP production, such as tomatidine, a steroidal plant alkaloid that was recently shown to target S. aureus ATP synthase subunit C (24).

Results from these studies support the concept that the mechanism of action of L-161, 982 is via inhibition of the electron transport chain and subsequent ATP production. Additional studies will establish the specific step or component of the electron transport chain inhibited by L-161, 982. This information can be important in determining species specificity of the compound. There is the possibility that L, 161, 982 may also exhibit antagonism with other classes of antibiotics. For example, S. aureus SCVs are perhaps better known as clinical isolates that display resistance to aminoglycosides, antibiotics that target the ribosomal 30S subunit and inhibit protein synthesis. Aminoglycosides require ATP for uptake by S. aureus, therefore inhibition of respiration is associated with drug resistance (23). For this reason, L-161, 982 may exert antagonism with this class of antibiotic. But with that withstanding, L-161, 982 is very effective against MRSA, both in vitro and in vivo (10). This is due to the fact that L, 161, 982 targets a completely different biological function than methicillin, which targets cell wall synthesis. Therefore, further development of electron transport chain inhibitors could represent a clinically important strategy to treat MRSA infections.

### References Cited in this Example

1. Sampedro G R, Bubeck Wardenburg J. 2017. Staphylococcus aureus in the Intensive Care Unit: Are These Golden Grapes Ripe for a New Approach? J Infect Dis 215:S64-S70.
2. Dantes R, Mu Y, Belflower R, Aragon D, Dumyati G, Harrison L H, Lessa F C, Lynfield R, Nadle J, Petit S, Ray S M, Schaffner W, Townes J, Fridkin S, Investigators EIPABCSMS. 2013. National burden of invasive methicillin-resistant Staphylococcus aureus infections, United States, 2011. JAMA Intern Med 173:1970-8.
3. Calfee D P. 2017. Trends in Community Versus Health Care-Acquired Methicillin-Resistant Staphylococcus aureus Infections. Curr Infect Dis Rep 19:48.
4. Gidengil C A, Gay C, Huang S S, Platt R, Yokoe D, Lee G M. 2015. Cost-effectiveness of strategies to prevent methicillin-resistant Staphylococcus aureus transmission and infection in an intensive care unit. Infect Control Hosp Epidemiol 36:17-27.
5. Nair N, Biswas R, Gotz F, Biswas L. 2014. Impact of Staphylococcus aureus on pathogenesis in polymicrobial infections. Infect Immun 82:2162-9.
6. Peters B M, Noverr M C. 2013. Candida albicans-Staphylococcus aureus polymicrobial peritonitis modulates host innate immunity. Infect Immun 81:2178-89.
7. Nash E E, Peters B M, Palmer G E, Fidel P L, Noverr M C. 2014. Morphogenesis is not required for Candida albicans-Staphylococcus aureus intra-abdominal infection-mediated dissemination and lethal sepsis. Infect Immun 82:3426-35.
8. Nash E E, Peters B M, Fidel P L, Noverr M C. 2015. Morphology-Independent Virulence of Candida Species during Polymicrobial Intra-abdominal Infections with Staphylococcus aureus. Infect Immun 84:90-8.
9. Ikeh M A C, Fidel P L, Noverr M C. 2018. Identification of Specific Components of the Eicosanoid Biosynthetic and Signaling Pathway Involved in pathological inflammation during Intra-abdominal Infection with. Infect Immun.
10. Ikeh M A C, Fidel P L, Jr., Noverr M C. 2018. Prostaglandin E2 Receptor Antagonist with Antimicrobial Activity against Methicillin-Resistant Staphylococcus aureus. Antimicrob Agents Chemother 62.
11. Carson C F, Mee B J, Riley T V. 2002. Mechanism of action of Melaleuca alternifolia (tea tree) oil on Staphylococcus aureus determined by time-kill, lysis, leakage, and salt tolerance assays and electron microscopy. Antimicrob Agents Chemother 46:1914-20.
12. Kohanski M A, Dwyer D J, Hayete B, Lawrence C A, Collins J J. 2007. A common mechanism of cellular death induced by bactericidal antibiotics. Cell 130:797-810.
13. Hitchings G H, Burchall J J. 1965. Inhibition of folate biosynthesis and function as a basis for chemotherapy. Adv Enzymol Relat Areas Mol Biol 27:417-68.
14. Zander J, Besier S, Saum S H, Dehghani F, Loitsch S, Brade V, Wichelhaus T A. 2008. Influence of dTMP on the phenotypic appearance and intracellular persistence of Staphylococcus aureus. Infect Immun 76:1333-9.
15. Proctor R A. 2008. Role of folate antagonists in the treatment of methicillin-resistant Staphylococcus aureus infection. Clin Infect Dis 46:584-93.
16. Mo C, Zhao R, Vallejo J, Igwe O, Bonewald L, Wetmore L, Brotto M. 2015. Prostaglandin E2 promotes proliferation of skeletal muscle myoblasts via EP4 receptor activation. Cell Cycle 14:1507-16.
17. Hosler J P, Yocum C F. 1987. Regulation of Cyclic Photophosphorylation during Ferredoxin-Mediated Electron Transport: Effect of DCMU and the NADPH/NADP Ratio. Plant Physiol 83:965-9.
18. Proctor R A, Dalal S C, Kahl B, Brar D, Peters G, Nichols W W. 2002. Two diarylurea electron transport inhibitors reduce Staphylococcus aureus hemolytic activity and protect cultured endothelial cells from lysis. Antimicrob Agents Chemother 46:2333-6.
19. Proctor R A, Kahl B, von Eiff C, Vaudaux P E, Lew D P, Peters G. 1998. Staphylococcal small colony variants have novel mechanisms for antibiotic resistance. Clin Infect Dis 27 Suppl 1:568-74.
20. Balwit J M, van Langevelde P, Vann J M, Proctor R A. 1994. Gentamicin-resistant menadione and hemin auxotrophic Staphylococcus aureus persist within cultured endothelial cells. J Infect Dis 170:1033-7.
21. Proctor R A, Balwit J M, Vesga O. 1994. Variant subpopulations of Staphylococcus aureus as cause of persistent and recurrent infections. Infect Agents Dis 3:302-12.
22. Chernyshev A, Fleischmann T, Kohen A. 2007. Thymidyl biosynthesis enzymes as antibiotic targets. Appl Microbiol Biotechnol 74:282-9.
23. von Eiff C, Peters G, Becker K. 2006. The small colony variant (SCV) concept—the role of staphylococcal SCVs in persistent infections. Injury 37 Suppl 2:S26-33.
24. Lamontagne Boulet M, Isabelle C, Guay I, Brouillette E, Langlois J P, Jacques P E, Rodrigue S, Brzezinski R, Beauregard P B, Bouarab K, Boyapelly K, Boudreault P L, Marsault E, Malouin F. 2018. Tomatidine Is a Lead Antibiotic Molecule That Targets Staphylococcus aureus ATP Synthase Subunit C. Antimicrob Agents Chemother 62.

### EQUIVALENTS

Those skilled in the art will recognize, or be able to ascertain, using no more than routine experimentation, numerous equivalents to the specific substances and procedures described herein. Such equivalents are considered to be within the scope of this invention, and are covered by the following claims.

What is claimed:

1. A method of preventing or treating a microbial infection in a subject, the method comprising administering to the subject an effective amount of an EP4 receptor antagonist.

2. The method of claim 1, wherein the EP4 receptor antagonist comprises L-161, 982 (CAS No. 147776-06-5), an analog thereof, or a derivative thereof.

3. The method of claim 1, wherein the EP4 receptor antagonist comprises a compound of the chemical structure:

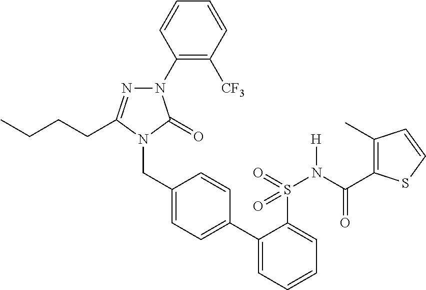 [SMILES: CCCCc1nn(-c2ccccc2C(F)(F)F)c(=O)n1Cc1ccc(-c2ccccc2S(=O)(=O)NC(=O)c2sccc2C)cc1]

4. The method of claim 1, wherein the EP4 receptor antagonist prevents or inhibits the growth of a microorganism.

5. The method of claim 1, wherein the EP4 receptor antagonist is administered prophylactically.

6. The method of claim 1, wherein the infection comprises a mono-species infection or a poly-species infection.

7. The method of claim 6, wherein the poly-species infection comprises a dual-species infection.

8. The method of claim 1, wherein the infection comprises a Staphylococcus infection, a Streptococcus infection, or both.

9. The method of claim 8, wherein the Staphylococcus infection comprises Staphylococcus aureus or Staphylococcus epidermidis.

10. The method of claim 8, wherein the Streptococcus infection comprises Streptococcus mutans.

11. The method of claim 9, wherein the infection comprises methicillin-resistant S. aureus (MRSA).

12. The method of claim 8, wherein the infection further comprises Candida.

13. The method of claim 12, wherein Candida comprises C. albicans.

14. The method of claim 6, wherein the poly-species infection comprises S. aureus and C. albicans.

15. The method of claim 1, wherein the microbial infection causes sepsis or peritonitis.

16. The method of claim 1, wherein the microbial infections comprises a bloodstream infection (i.e., bacteremia).

17. The method of claim 1, wherein the EP4 receptor antagonist is administered at a dose of about 10 mg/kg body weight.

18. The method of claim 1, wherein the EP4 receptor antagonist is administered at a dose of about 6.3 μg/ml.

19. The method of claim 1, wherein the EP4 receptor antagonist is provided in a pharmaceutically acceptable composition or as a pharmaceutically acceptable salt.

20. The method of claim 1, wherein the EP4 receptor antagonist is administered topically or systemically.

21. A method of inhibiting the growth of a microorganism, the method comprising contacting the microorganism with an EP4 receptor antagonist for a period of time sufficient to inhibit the growth of the microorganism.

22. The method of claim 21, wherein the EP4 receptor antagonist comprises L-161, 982 (CAS No. 147776-06-5), an analog thereof, or a derivative there.

23. The method of claim 21, wherein the EP4 receptor antagonist comprises a compound of the chemical structure:

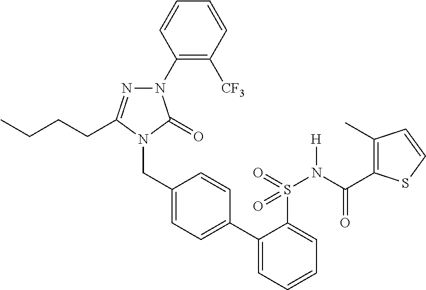 [SMILES: CCCCc1nn(-c2ccccc2C(F)(F)F)c(=O)n1Cc1ccc(-c2ccccc2S(=O)(=O)NC(=O)c2sccc2C)cc1]

24. A method of preventing biofilm formation, the method comprising applying to a surface of interest an antimicrobial composition comprising an EP4 receptor antagonist.

25. The method of claim 24, wherein the surface comprises the surface of a medical device.

26. The method of claim 25, wherein the medical device is coated with the EP4 receptor antagonist.

27. The method of claim 24, wherein the composition comprises a disinfectant.

28. The method of claim 27, wherein the disinfectant comprises an aerosol.

29. A method of preventing biofilm formation, the method comprising incorporating into a material an EP4 receptor antagonist.

30. The method of claim 29, wherein the material comprises a bio-compatible material.

31. A method of disrupting a biofilm, the method comprising contacting the microbial biofilm with an EP4 receptor antagonist for a period of time sufficient to disrupt the pre-formed microbial biofilm.

32. The method of claim 24 or claim 29, wherein the EP4 receptor antagonist comprises L-161, 982 (CAS No. 147776-06-5), an analog thereof, or a derivative there.

33. The method of claim 24 or claim 29, wherein the EP4 receptor antagonist comprises a compound of the chemical structure:

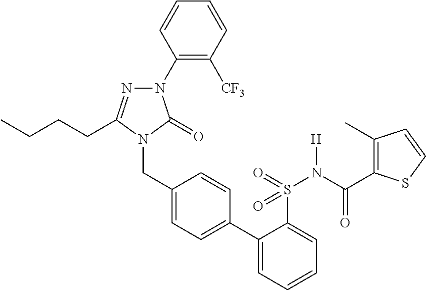 [SMILES: CCCCc1nn(-c2ccccc2C(F)(F)F)c(=O)n1Cc1ccc(-c2ccccc2S(=O)(=O)NC(=O)c2sccc2C)cc1]

34. The method of claim 24 or claim 29, wherein the biofilm comprises a mono-species biofilm or a poly-species biofilm.

35. The method of claim 34, wherein the poly-species biofilm comprises a dual-species bio-film.

36. The method of claim 24 or claim 29, wherein the biofilm comprises Staphylococcus, Streptococcus, or both.

37. The method of claim 36, wherein Staphylococcus comprises Staphylococcus aureus or Staphylococcus epidermidis.

38. The method of claim 36, wherein the Streptococcus comprises Streptococcus mutans.

39. The method of claim 36, wherein the biofilm further comprises Candida.

40. The method of claim 39, wherein the Candida comprises C. albicans.

41. The method of claim 36, wherein the biofilm comprises S. aureus and C. albicans.

42. The method of any one the above claims, wherein the microorganism comprises a bacterium.

43. The method of claim 42, wherein the bacterium comprises a gram-positive bacterium.

44. The method of claim 42, wherein the bacterium comprises antibiotic-resistant bacterium.

45. The method of claim 44, wherein the antibiotic comprises methicillin.
